(19) United States
(12) Patent Application Publication
(10) Pub. No.: US 2018/0284131 A1
(43) Pub. Date: Oct. 4, 2018

(54) DIAGNOSTIC ASSAYS FOR SUPAR-&#946;3 INTEGRIN DRIVEN KIDNEY DISEASES

(71) Applicants: THE GENERAL HOSPITAL CORPORATION, Boston, MA (US); RUSH UNIVERSITY MEDICAL CENTER, Chicago, IL (US)

(72) Inventors: Sanja Sever, Brookline, MA (US); Jochen Reiser, Hinsdale, IL (US)

(21) Appl. No.: 15/763,016

(22) PCT Filed: Sep. 26, 2016
(86) PCT No.: PCT/US16/53788

§ 371 Date: Mar. 23, 2018

Related U.S. Application Data
(60) Provisional application No. 62/232,606, filed on Sep. 25, 2015.

(51) Int. Cl. G01N 33/68; C07K 16/28; G01N 33/50

(52) U.S. Cl. CPC G01N 33/6872; C07K 16/2848; G01N 33/6869; G01N 2333/70557; G01N 2800/347; G01N 2333/70596; G01N 2333/5412; G01N 33/5005

(57) ABSTRACT

Methods for diagnosing suPAR-β3 integrin driven kidney diseases that can include detection of one, two or more variables, e.g., biomarkers (plasma suPAR levels, urine IL6 levels) and/or bioassays (β3 integrin activation and presence of distinct suPAR fragments/isoforms in plasma).

### CLAIM OF PRIORITY

This application claims the benefit of U.S. Patent Application Ser. No. 62/232,606, filed on Sep. 25, 2015. The entire contents of the foregoing are hereby incorporated by reference.

### FEDERALLY SPONSORED RESEARCH OR DEVELOPMENT

This invention was made with Government support under Grant No. 1 R01 DK101350 awarded by the National Institutes of Health. The Government has certain rights in the invention.

### TECHNICAL FIELD

Described herein are methods for diagnosing and treating Chronic kidney diseases (CKDs), e.g., suPAR-β3 integrin driven kidney diseases. The methods can include detection of one, two or more variables, e.g., biomarkers (plasma suPAR levels, urine IL6 levels) and/or bioassays (β3 integrin activation and presence of distinct suPAR fragments/isoforms in plasma).

### BACKGROUND

Chronic kidney diseases (CKDs), a progressive loss of renal function over a period of months or years, affects hundreds of millions of people worldwide. The three most common causes of CKD are diabetes mellitus, hypertension, and glomeruloneprhritis. Historically, kidney disease has been classified according to the part of the renal anatomy involved such as vascular disease, glomerular disease, and tubulointerstitial disease. Glomerular disease comprises a diverse group and is classified into primary glomerular disease such as focal segmental glomerulosclerosis (FSGS) and IgA nephropathy, and secondary glomerular disease such as diabetic nephropathy and lupus nephritis.

While major progress has been made in identifying genetic mutations that underlie hereditary forms of FSGS (1-4), kidney biopsy is still widely used for diagnosing glomerular diseases.

### SUMMARY

It has been suggested that suPAR drives kidney injury by activating β3 integrin on podocytes, thus providing a potential downstream pathogenic pathway that could be used to develop kidney-specific diagnostic tools.

Soluble urokinase-type plasminogen activator receptor (suPAR) is the soluble form of the urokinase-type plasminogen activator receptor (uPAR) and is present in plasma and other body fluids. suPAR has different biological forms and is being evaluated as an inflammatory and life style risk biomarker. suPAR was recently identified as a risk factor for both onset as well as progression of CKD regardless of its entomology (5, 6). Elevated suPAR levels in the serum of the patients were originally implicated as a specific risk factor for recurrent FSGS (5), but subsequent studies showed elevated suPAR levels and its association with diabetic nephropathy in patients with type 1 diabetes (see below and 7). Taking into account that suPAR levels could also be elevated due to loss of kidney function (8), and since elevated suPAR levels are associated with diverse pathogenic conditions such as sepsis, cancer (see, e.g., Sier et al., Thromb Haemost. 2004 February; 91(2):403-11, which found low molecular weight suPAR (e.g., D2D3) in the urine of both healthy and cancer patients, but none in their serum), and coronary artery disease, in addition to diverse chronic kidney diseases such as focal segmental glomerulosclerosis (FSGS) and diabetic nephropathy (DN), additional diagnostics could provide more specificity to diagnose suPAR-driven CKD in patients that already have impaired kidney function.

It has been suggested that suPAR drives kidney injury by activating 133 integrin on podocytes, thus providing a potential downstream pathogenic pathway that could be used to develop kidney-specific diagnostic tools. Given the potential of suPAR as a novel therapeutic target in CKD, it was hypothesized that a combination of suPAR levels together with assays that detect suPAR-driven podocyte injury might allow development of the novel molecular diagnostic tools in nephrology that would be able to diagnose suPAR-β3 integrin driven pathogenic mechanism in CKD in general, and recurrent FSGS in particular. With novel therapeutics that target β3 integrin currently being developed and tested in humans (Maile et al., J Diabetes Res. 2014; 2014:421827; and Maile et al., Endocrinology. 2014 December; 155(12):4665-75), a diagnostic tool that can specifically detect β3 integrin-driven glomerular injury would be helpful in obtaining meaningful results from human trials and for patients with CKD.

Thus, provided herein are methods comprising two, three, or all four of the following (in any order): determining a level of soluble urokinase-type plasminogen activator receptor (suPAR) protein in a plasma sample from a subject; determining a level of interleukin 6 (IL-6) protein in a urine sample from the same subject; determining a level of low molecular weight suPAR in a plasma sample from the subject; and determining a level of β3 integrin activation activity in a plasma sample from the subject.

Also provided herein are methods for detecting the presence of suPAR-β3 integrin driven kidney disease in a subject. The methods include (a) determining a subject level of two, three, or all four of the following markers (in any order): soluble urokinase-type plasminogen activator receptor (suPAR) protein in a plasma sample from the subject; interleukin 6 (IL-6) protein in a urine sample from the subject; low molecular weight suPAR in a plasma sample from the subject; and β3 integrin activation activity in a plasma sample from the subject; (b) comparing the subject level of the marker to a reference level; and (c) detecting the presence of suPAR-β3 integrin driven kidney disease in a subject who has at least two markers above the level.

Further provided herein are methods for treating a subject who has chronic kidney disease, comprising (a) determining a subject level of two, three, or all four of the following markers (in any order): soluble urokinase-type plasminogen activator receptor (suPAR) protein in a plasma sample from the subject; interleukin 6 (IL-6) protein in a urine sample from the subject; low molecular weight suPAR in a plasma sample from the subject; and β3 integrin activation activity in a plasma sample from the subject; (b) comparing the subject level of the marker to a reference level; and (c) selecting and optionally administering a treatment for suPAR-β3 integrin driven kidney disease to a subject who has at least two markers above the level.

In some embodiments, detecting β3 integrin activation activity in a plasma sample comprises contacting the plasma sample with cultured human podocytes in vitro and determining a level of β3 integrin activation in the sample.

In some embodiments, determining a level of β3 integrin activation in the sample comprises contacting the sample with an antibody that binds to beta 3 integrin and an antibody that binds to paxillin and dividing the number of cells expressing beta 3 integrin by the number of cells expressing paxillin.

In some embodiments, the subject has chronic kidney disease.

In some embodiments, the reference value is serum suPAR of ≥3 ng/ml (e.g., determined by ELISA assay); β3 integrin activation >1.2 (e.g., determined by AP5/paxillin ratio normalized to healthy serum); presence of low molecular weight suPAR in serum (e.g., determined by a detectable band on Western blot analysis; the detection limit of the assay may depend on the efficacy of the immunoprecipitation procedure); detectable presence of IL6 in the urine (e.g., determined by ELISA assay, e.g., wherein only a positive signal in ELISA (above the background) is considered positive).

In some embodiments, the methods include determining a score calculated using the following algorithm:

score=α×(serum suPAR)+β×(β3 integrin activation)+γ×(low molecular weight suPAR)+δ×(urine IL-6),

wherein each of a, β, γ, and δ are empirically determined weights. In some embodiments, the algorithm is:

score=0.253×(serum suPAR)+0.282×(β3 integrin activation)+0.212×(low molecular weight suPAR)+0.253×(urine IL-6).

In some embodiments, the methods include selecting and/or administering a treatment for suPAR-β3 integrin driven kidney disease, e.g., an α5β3 inhibitor and/or ex vivo removal of suPAR from the subject's circulation. In some embodiments, the α5β3 inhibitor is a monoclonal antibody that binds specifically to α5β3; a peptide comprising a RGD binding sequence; or a small molecule α5β3 inhibitor. In some embodiments, the small molecule α5β3 inhibitor is a compound of the formula

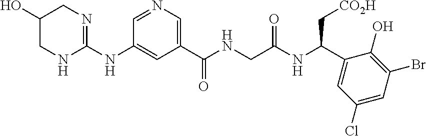 [SMILES: O=C(O)C[C@H](NC(=O)CNC(=O)c1cncc(NC2=NCC(O)CN2)c1)c1cc(Cl)cc(Br)c1O]

or a pharmaceutically acceptable salt thereof.

In some embodiments, the monoclonal antibody that binds specifically to α5β3 is VPI-2960B, CNTO95, or anti-CD61.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F. suPAR levels in plasma do not correlate with β3 integrin activation.

A) SuPAR levels in serums of patients with different etiologies: healthy individuals, focal segmental glomerulosclerosis (FSGS), diabetic nephropathy (DN), on dialysis due to end stage renal diseases, sepsis.

B-F) Graphs correlating suPAR level in serum with β3 integrin activation determined as a ratio between AP5 and paxillin staining. Percent of serums that exhibit β3 integrin activation is shown by number and darker color in a Pie Chart.

FIGS. 2A-C. Immunofluorescence assay that measures β3 integrin activation using human plasma.

A) Immunofluorescence (IF) staining of human podocytes incubated with either healthy serum or FSGS serum. Cells were stained for paxillin (marker of focal adhesions) and activated β3 integrin using AP5 antibody from Bood Center of Wisconsin.

B) Graph showing β3 integrin activation by serum free media (SFM), media with fetal bovine serum (FBS), or serums (#1-4) from healthy individuals. β3 integrin activation is determined as a ratio between AP4 and paxillin staining.

C) Representative images of IF analysis using ImageJ program.

FIGS. 3A-G. D2D3 fragment is potent activator of β3 integrin on podocytes and it can be detected in subset of patient serums.

A) Integrin activation of human podocytes treated with healthy serum, FSGS serum (control), FSGS serum incubated with anti-suPAR Ab, FSGS serum from which suPAR was immune precipitated using anti-suPAR Ab (ΔsuPAR), or that was incubated with only Protein G beads.

B) Western blot analysis of suPAR in human serums before and after they were incubated with Protein G or Protein A beads. Data show that suPAR from the human serum binds Protein G or Protein A beads, even in the absence of specific anti-suPAR antibody.

C) Western blot analysis of glycosylated and de-glycosylated suPAR in human serum.

D) Western blot analysis of D2D3 fragment present in human serum sample #2.

E) Integrin activation detected in human podocytes incubated with serum free media (SFM), FSGS serum, serum free media to which 2 ng/ml of suPAR or D2D3 was added. Mn2+ was used as non-specific control of integrin activation.

F) Integrin activation detected in human podocytes incubated with healthy serum (HS), FSGS serum and healthy serum with added 2 ng/ml of suPAR or D2D3 fragment.

G) D2D3 exhibits cooperative behavior with regard to β3 integrin activation, in contrast to full length suPAR that activates β3 integrin at much higher concentrations.

FIGS. 4A-G. Integrin activation does not lead to increase in mRNA for integrins.

A) Schematic diagram of domains that constitute full length suPAR and D2D3 fragment used in this study.

B) Silver staining gel of recombinant suPAR from R&D, D2D3 fragment expressed and purified from either bacteria or insect cells, as indicated in the figure.

C) PLAUR Ab recognizes both suPAR and D2D3 by Western blot analysis.

D,E) RT-PCR of mRNA encoding αv, β3, α3, β1 integrins in the presence of suPAR or D2D3 fragment.

F,G) RT-PCR of mRNA encoding αv, β3, α3, β1 integrins in the presence of different serums. No treatment altered expression levels of above integrins.

FIGS. 5A-F. D2D3 induces podocyte motility

A-C) High throughput assays determining β3 integrin activation at focal adhesions (A), in the cytoplasm (B), or measuring total cell area (C).

D) High throughput assay examining podocyte motility in the presence of indicated concentrations of D2D3. LPS treatment was used as a positive control.

E) Bar graph showing podocyte motility under conditions shown in (D).

F) Podocytes viewed by lower magnification, show that at higher D2D3 concentration (25 ng/ml) cells start to detach from the coverslip.

FIGS. 6A-J. D2D3 fragment induces proteinuria and glomerular injury in mice.

A-C) Bar graphs showing proteinuria (kidney injury) or the lack of in animals injected with PBS (A, control), human suPAR (B), or human D2D3 (C). Only injection of D2D3 resulted in transient proteinuria in mice. N=6 animals.

D) Western blot analysis of urine from animals injected with PBS or D2D3. Data show presence of nephrin (podocyte specific protein) in the urine of proteinuric animals, suggesting podocyte injury.

E and H) Schematic diagrams of suPAR transgenes used in this study.

F and I) Graphs showing levels of albumin/creatinine (proteinuria) in mice stably expressing mouse D2D3 (F) or Isoform 2 (I) from fat tissue. Animals were fed with high fat diet to induce protein expression.

G and J) Representative image of glomerulus stained with PAS. Image shows that glomerulus exhibits signs of injured glomerulus similar to that observed during diabetic nephropathy.

FIGS. 7A-B. Alignment of protein sequences encoding uPAR variants as indicated in the Figure.

FIGS. 8A-D. Human isoform 2 induces podocyte detachment.

A) Immunofluorescence (IF) staining of human podocytes incubated with healthy serum (Control), Mn2+ or increasing concentrations of human suPAR variant 2. Cells were stained for paxillin (marker of focal adhesions) and activated β3 integrin using AP5 antibody from Blood Center of Wisconsin.

B-D) Bar graphs showing integrin activation (B), number of cells (C) and number of focal adhesions (FA) per cell (D) in cells treated as shown in (A).

FIG. 9. Table showing representative data sets of 18 FSGS and 5 healthy serums. We originally determined a value for each of the parameters using logistic regression analysis, but that analysis as shown in the Figure determined a similar value for each one of the parameters. Thus, we decided to use a total score (form 0-4) instead of the specific value determined using regression analysis.

FIG. 10. ROC (receiver operating characteristic) curves with 4 different parameters, which separate recurrent from non-recurrent FSGS. Area under the curve was calculated for each one of 4 parameters using ROC analysis in order to separate subjects with recurrent FSGS from non-recurrent FSGS. Area under the curve that is >0.5 is considered statistically significant. Of note, we originally also determined the levels of suPAR in patient's urine, but since the ROC analysis did not show statistically significant area under the curve value (see enclosed Figure, value was 0.327), that parameter was not used in generating the composite score assay. Data shown were generated by using 22 recurrent and 7 non-recurrent FSGS serums.

FIG. 11. Composite score of ≥3 efficiently not only separates recurrent from non-recurrent FSGS but is also identifying suPAR-β3 integrin pathway in population suffering from DN. Positive value (+) was assigned to: 1. serum suPAR of ≥3 ng/ml (Elisa assay); 2. β3 integrin activation >1.2 (AP5/paxillin ratio); 3. presence of D2D3 in serum determined by Western blot analysis; 4. detectable presence of IL6 in the urine (ELISA assay). Data were generated using 29 FSGS and 32 DN samples.

FIGS. 12 A-D. ROC (receiver operating characteristic) curves with 3 different parameters. Area under the curve was calculated for each one of the 3 indicated parameters (Score 1); combination of any two parameters (Score 3) and finally the combination of tree parameters (Score 3). FIG. 12A is a combination of the suPAR, AP5, and IL6 parameters. FIG. 12B is a combination of the suPAR, AP5, and low molecular weight suPAR parameters. FIG. 12C is a combination of the suPAR, low molecular weight suPAR, and IL6 parameters. FIG. 12D is a combination of the AP5, low molecular weight suPAR, and IL6 parameters.

### DETAILED DESCRIPTION

The selectivity of the glomerular filter is maintained by physical, chemical, and signaling interplay among its three core constituents—the glomerular endothelial cells, the glomerular basement membrane (GBM), and podocytes. Injury to or functional impairment of any of these three components of the glomerular filtration barrier can lead to proteinuria (11). Podocytes are injured in many forms of human and experimental glomerular disease, including minimal change disease, focal segmental glomerulosclerosis (FSGS), and diabetes mellitus (12). Podocytes are terminally differentiated visceral epithelial cells of the glomerulus which develop a characteristic architecture specialized for glomerular ultrafiltration. Their structure is traditionally divided into three kinds of subcellular compartment: the cell body, microtubule-driven membrane extensions named primary process, and actin-driven membrane extensions named foot processes (FPs). Adjacent podocytes are interdigitated with each other at their foot processes, which are separated from each other by filtration slits and bridged with a specialized intercellular junction called a slit diaphragm. The foot processes and slit diaphragm serve as an adhesive apparatus to the glomerular basement membrane (GBM), which together with endothelial cells and their glycocalyx forms a filtration barrier.

Regardless of the underlying cause of glomerular disease, the early pathogenic events are characterized by molecular alterations in the slit diaphragm without visible morphological changes or, more obviously, by a reorganization of the FPs structure with fusion of filtration slits termed “FP effacement” (12-14). Although it is possible to have proteinuria without significant FP effacement, for over 50 years FPs effacement has been a cardinal feature of proteinuria. While the mechanistic significance of FPs effacement with regard to proteinuria has long been a mystery, over the last decade numerous studies demonstrated that FPs effacement represents a change in the organization of the actin cytoskeleton (12, 15).

As noted above, one of the pathogenic pathways implicated in CKD is suPAR-β3 integrin pathway (Wei et al., Nature medicine. 2011; 17(8):952-60; Wei et al., Nat Med. 2008 January; 14(1):55-63). By determining the ability of the low molecular weight suPAR as well as splice variant 2 to induce proteinuria and glomerular injury in mice via activating β3 integrin on podocytes, the data presented herein establish methods including a composite scoring system that efficiently separate non-recurrent from recurrent FSGS. The same scoring system also identified a subset of patients with DN as positive subjects. Given current and future attempts to develop novel therapeutic targets for CKD, and given that CKD encompasses a highly diverse group of patients, it is become more and more important to identify patients that are predicted to respond to target-specific therapy. The methods described herein can be used to identify subjects that have suPAR-β3 integrin driven kidney diseases, and thus are expected to respond (i.e., have a stabilized or improved condition) to either suPAR and/or β3 blocking therapies.

Described herein are methods that use a combination of biomarkers (plasma suPAR levels, urine IL6 levels) and/or bioassays (β3 integrin activation and presence of low molecular weight suPAR, e.g., fragments/isoforms, in plasma) together to generate a diagnostic score that predicts β3-integrin driven kidney injury. Unexpectedly, as shown herein, a positive score was associated with a majority of patients with recurrent FSGS and with a subset of patients with DN.

Subjects

In the present methods a subject who may be evaluated using the present methods can be a human or other mammal, typically one who is at risk of developing or has a disorder characterized by proteinuria, is at risk for or is undergoing kidney failure, has received a kidney graft, or any combination thereof. A disorder characterized by proteinuria includes, for example, kidney or glomerular diseases (e.g., FSGS), membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis, IgA nephropathy, IgM nephropathy, membranoproliferative glomerulonephritis, membranous nephropathy, end-stage kidney disease, sarcoidosis, Alport's syndrome, diabetes mellitus (e.g., diabetic nephropathy), kidney damage due to drugs, Fabry's disease, infections, aminoaciduria, Fanconi syndrome, hypertensive nephrosclerosis, interstitial nephritis, sickle cell disease, hemoglobinuria, multiple myeloma, myoglobinuria, Wegener's granulomatosis, and glycogen storage disease type 1. In some embodiments, the subject may be affected by one or more of the foregoing disorders, may be a heterozygote for the polymorphism Leu33Pro in the human integrin β3 gene, may be a homozygote for the polymorphism Leu33Pro in the human integrin β3 gene, may have at least about 3 ng suPAR per ml blood in the circulation, or any combination thereof.

In some embodiments, subjects who may be evaluated using the present methods include those who have chronic kidney disease (CKD) or are at risk of developing CKD, e.g., who have acute kidney injury (AKI) or another condition noted above, e.g., a disorder characterized by proteinuria.

The stages of CKD are classified as follows:

Stage 1: Kidney damage with normal or increased GFR (>90 mL/min/1.73 m 2);

Stage 2: Mild reduction in GFR (60-89 mL/min/1.73 m²);

Stage 3a: Moderate reduction in GFR (45-59 mL/min/1.73 m²);

Stage 3b: Moderate reduction in GFR (30-44 mL/min/1.73 m²);

Stage 4: Severe reduction in GFR (15-29 mL/min/1.73 m²); and

Stage 5: Kidney failure (GFR <15 mL/min/1.73 m² or dialysis).

In stage 1, given the relatively normal GFR, a diagnosis may be confirmed by the presence of one or more of the following:

Albuminuria (albumin excretion >30 mg/24 hr or albumin:creatinine ratio >30 mg/g [>3 mg/mmol]);

Urine sediment abnormalities;

Electrolyte and other abnormalities due to tubular disorders;

Histologic abnormalities;

Structural abnormalities detected by imaging; or

History of kidney transplantation.

See, e.g., Kidney Disease: Improving Global Outcomes (KDIGO) CKD Work Group. KDIGO 2012 Clinical Practice Guideline for the Evaluation and Management of Chronic Kidney Disease. Kidney Int Suppl. 2013. 3:1-150. Standard laboratory and clinical methods can be used to establish a diagnosis.

Focal segmental glomerulosclerosis (FSGS) is a significant cause of end-stage kidney disease. It affects both native kidneys and transplanted kidney grafts. It starts in kidney glomeruli. In the early stage of FSGS, it mainly targets the visceral epithelium (also called podocytes) that comprise cells with foot processes to regulate functioning of the renal filtration barrier. Effacement of podocyte foot processes can mark the first or one of the first ultrastructural step(s) that is/are associated with loss of plasma proteins into the urine. While gene defects in podocytes have been identified for hereditary FSGS, there are also cases that occur in the absence of gene defects or with post-transplant recurrence in about 30% of patients receiving a kidney graft. These observations led to the suggestion that development of FSGS can be associated with a “FSGS permeability factor” in the patient's circulation (see Savin et al., Translational Res. 151:288-292, 2008). Without wishing to be bound by theory, suPAR is likely to be that factor; see WO 2010/054189 and WO 2012/154218.

Methods of Diagnosis and Monitoring

Included herein are methods for diagnosing suPAR-β3 integrin driven kidney diseases. The methods include detection of one, two or more variables, e.g., biomarkers (plasma suPAR levels, urine IL6 levels) and/or bioassays (β3 integrin activation and presence of distinct lower molecular weight suPAR fragments/isoforms in plasma). The methods can include obtaining or providing a plasma and/or urine sample from a subject, and determining one, two, three, or all four of the following variables: (1) the presence and/or level of total suPAR in the plasma; (2) the presence and/or level of low molecular weight suPAR (e.g., suPAR fragments/isoforms that are not full length) in the plasma; (3) presence and/or levels of IL6 in the urine; and/or (4) presence and/or levels of β3 integrin activation (e.g., in an in vitro assay).

The low molecular weight suPAR detected in the present methods include those that are not full length suPAR, and thus have a lower molecular weight than full length suPAR. Full length suPAR is approximately 50 kD when fully glycosylated and approximately 32.5 kD when deglycosylated. The low molecular weight suPAR fragments/isoforms can include those that lack D1 (e.g., as a result of proteolysis, e.g., D2D3 fragment) or are splice variants, e.g., suPAR2. The low molecular weight suPAR fragments/isoforms have a molecular weight of less than 50 kD, e.g., about 25-45 kD, e.g., about 30 kD, when fully glycosylated and less than 32.5 kD, e.g., about 20-30 kD, e.g., about 25 kD when deglycosylated. In this context, “about” means±10%.

As used herein the term “sample”, when referring to the material to be tested for the presence of a biological marker using the method of the invention, unless otherwise specified can include inter alia tissue, whole blood, plasma, serum, urine, sweat, saliva, breath, exosome or exosome-like microvesicles (U.S. Pat. No. 8,901,284), lymph, feces, cerebrospinal fluid, ascites, bronchoalveolar lavage fluid, pleural effusion, seminal fluid, sputum, nipple aspirate, post-operative seroma or wound drainage fluid. The type of sample used may vary depending upon the identity of the biological marker to be tested and the clinical situation in which the method is used. Various methods are well known within the art for the identification and/or isolation and/or purification of a biological marker from a sample. An “isolated” or “purified” biological marker is substantially free of cellular material or other contaminants from the cell or tissue source from which the biological marker is derived, i.e., partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a protein (e.g., of IL-6, suPAR total, and/or low molecular weight suPAR, e.g., suPAR fragments and isoforms) can be evaluated using methods known in the art, e.g., using standard electrophoretic and quantitative immunoassay methods for proteins, including but not limited to, Western blot, e.g., with immunoprecipitation of specific proteins; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance. Antibodies to suPAR and IL-6 are known in the art and commercially available. Antibodies that bind specifically to low molecular weight suPAR can be generated using known methods (see, e.g., Sier et al., Thromb Haemost. 2004 February; 91(2):403-11). See also WO2012154218 for additional information on measuring levels of suPAR in serum.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically, a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention. (See U.S. Pat. Nos. 5,118,937; 5,045,694; 5,719,060; 6,225,047). Such methods may be particularly useful for determining presence and/or levels of low molecular weight suPAR fragments and isoforms.

Integrin activation can be determined by an assay such as the one described herein. In this exemplary assay, β3 integrin activation on podocytes is measured in vitro. The podocytes are cultured in the presence of serum from the subject for a time sufficient to allow activation of integrin and/or development of focal adhesions (e.g., approximately 0.1-10%, approximately 5-10%, or approximately 10% human serum in media; for example, 1 ml media will have 0.1 ml of human serum; time can be approximately 12 to 24 hours, or approximately 24 hours) and the ratio of activated integrin levels over levels of total amount of focal adhesions is determined. Activated integrin levels can be determined, e.g., using an antibody that specifically detects the active form of β3 integrin, e.g., AP5 (see, e.g., Honda et al., 270(20):11947-54 (1995); Faccio et al., Journal of Cell Science 115, 2919-2929 (2002); and Wei et al., Nature Medicine 17:952-960 (2011). AP5 antibodies are available commercially from Kerafast (Boston, Mass.). The number of focal adhesions can be determined using known methods, e.g., by staining with paxillin and determining the number or level of focal adhesions within a selected field.

In the present methods, two, three, or more of the variables can be determined, e.g., as follows:

| | | Levels of low | |
|---|---|---|---|
| Levels of total | Levels of IL-6 | molecular weight | Integrin |
| suPAR in plasma | in urine | suPAR in plasma | activation |
| X | X | | |
| X | | X | |
| X | | | X |
| | X | X | |
| | X | | X |
| | | X | X |
| X | X | X | |
| | X | X | X |
| X | | X | X |
| X | X | | X |
| X | X | X | X |

The methods can include comparing the presence and/or level of each variable with one or more references, e.g., a control reference that represents a normal level, e.g., a level in an unaffected subject, and/or a disease reference that represents a level associated with suPAR-β3 integrin driven kidney diseases, e.g., a level in a subject having FSGS, e.g., a subject having an increased risk of reoccurrence of FSGS after kidney transplant. Methods of determining threshold or reference levels are known in the art, and exemplary methods are described herein. In some embodiments, the threshold levels are: suPAR in the serum above 3 ng/ml; β3 integrin activation above 1.2; presence of any detectable low molecular weight suPAR if determined by fragment IP followed by the western blot (e.g., a level above the lowest level of detection for a standard assay); presence of any detectable levels of IL6 in the urine (e.g., a level above the lowest level of detection for a standard assay). In some embodiments, the methods also include detecting suPAR in the urine (with an exemplary threshold of 3 ng/ml or above).

The methods can also include calculating a score based on the variables that can be compared to a reference score, wherein a score that is above the reference score indicates that the subject has suPAR-β3 integrin driven kidney disease and/or a high risk of, or is likely to have, recurrence of kidney disease after transplant, or is predicted to have a positive response to therapy targeting suPAR-β3 integrin pathway; a score below the reference score indicates that the subject has a low risk of recurrence of disease after transplant, or is predicted to have no or a poor response to therapy targeting suPAR-β3 integrin pathway. a “high” risk as used herein indicates that the subject has a statistically increased (e.g., at least greater than 50%) chance of recurrence or response as compared to someone with a score associate with a “low” risk.

In some embodiments, the levels of each of the evaluated variables can be assigned a value (e.g., a value that represents the level of the biomarker or activation level, e.g., normalized as described herein). For example, value of 0 or 1 can be assigned to each of the evaluated parameters. That value (optionally weighted to increase or decrease its effect on the final score) can be summed or otherwise mathematically manipulated to produce a final score. One of skill in the art could optimize such a method to determine an optimal algorithm for determining a score; one exemplary method is described herein.

For example, a weighted average formula can be used to generate a composite score assay. In some embodiments, a composite score can be calculated based on all four of the variables, using the following algorithm:

score=α×(serum suPAR)+β×(β3 integrin activation)+γ×(low molecular weight suPAR)+δ×(urine IL-6)

Wherein each of α, β, γ, and δ are empirically determined weights. An exemplary formula with weights included can be:

score=0.253×(serum suPAR)+0.282×(β3 integrin activation)+0.212×(low molecular weight suPAR)+0.253×(urine IL-6)

Threshold levels can be determined empirically. One of skill in the art will appreciate that references can be determined using known epidemiological and statistical methods, e.g., by determining a score, or protein or activation levels, in an appropriately stratified cohort of subjects, e.g., subjects who have or do not have a recurrence of disease after transplant.

In exemplary embodiments, the thresholds can be as follows: suPAR in the serum above 3 ng/ml; β3 integrin activation above 1.2; presence of any detectable low molecular weight suPAR if determined by fragment IP followed by the western blot (e.g., a level above the lowest level of detection for a standard assay); presence of any detectable levels of IL6 in the urine (e.g., a level above the lowest level of detection for a standard assay). A score above a certain level, e.g., a score of >0.5 or >0.7, can be considered positive for suPAR-β3 integrin driven podocyte injury.

The threshold level for each variable or for the overall score can be determined using known epidemiological and statistical methods; in some embodiments the level can be, e.g., a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

As noted above, any of the two variables evaluated herein can be used, rather than all four, with reasonably good sensitivity; using additional variables increases the specificity. In some embodiments, all 4 variables are evaluated, and a positive score on any two, or any 3 in combination (score of 0.5 or 0.7) will be very specific for suPAR-b3 pathway. However, depending on the specificity desired, a subset can also be used. When two are used, the presence of two positive results indicates can be considered positive for suPAR-β3 integrin driven podocyte injury. When three are used, the presence of two or three positive results indicates can be considered positive for suPAR-β3 integrin driven podocyte injury.

Subjects associated with predetermined values are typically referred to as reference subjects. For example, in some embodiments, a control reference subject does not have a disorder described herein (e.g., does not have suPAR-β3 integrin driven kidney disease or does not have a recurrence of disease after kidney transplant). In some cases, it may be desirable that the control subject has kidney disease (e.g., FSGS), and in other cases it may be desirable that a control subject has no kidney disease. In some cases, it may be desirable that the control subject has a recurrence of disease after transplant and in other cases it may be desirable that a control subject does not have a recurrence after transplant.

Thus, in some cases the variable in a subject being greater than or equal to a reference variable is indicative of a clinical status (e.g., indicative of a disorder as described herein, e.g., suPAR-β3 integrin driven kidney disease or high risk of recurrence after transplant). In other cases, the level of the variable in a subject being less than or equal to the reference variable is indicative of the absence of suPAR-β3 integrin driven kidney disease or normal or low risk of recurrence. In some embodiments, the amount by which the level in the subject is the less than the reference level is sufficient to distinguish a subject from a control subject, and optionally is a statistically significantly less than the level in a control subject. In cases where the variable in a subject being equal to the reference variable, the “being equal” refers to being approximately equal (e.g., not statistically different).

The predetermined value can depend upon the particular population of subjects (e.g., human subjects) selected. For example, an apparently healthy population will have a different ‘normal’ range of levels of the measured variables than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

The present methods can also be performed multiple times on the same subject, e.g., before, after, and during treatment, to monitor the effectiveness of the treatment, or without any treatment, e.g., to monitor the subject's condition (e.g., the severity of their disease). An increase in levels of the measured markers and/or activation over time indicates that the subject's condition is worsening (for example, progressing from acute kidney injury (AKI) to CKD); no change in levels means that the subject is stable (in a subject with progressive disease, this may indicate that the treatment has stabilized the disease); and a decrease indicates that the subject's condition is improving (e.g., the treatment is effective). The methods can be used to determine if or when to begin treatment, for example, when a subject has progressed to severe enough disease to warrant further intervention. The methods can be used to select a treatment; for example, the methods can be used to select subjects who would be most likely to benefit from a treatment directed at affecting suPAR-β3 integrin driven pathogenesis; those with higher levels (e.g., levels above a threshold) of the measured markers and/or activation would be more likely to benefit. Furthermore, the methods can be used to identify those who are most likely to have a relapse of FSGS after transplant (i.e., those with higher levels (e.g., levels above a threshold) of the measured markers and/or activation), and are thus better candidates for a cadaver organ rather than from a living donor.

Methods of Treatment

The methods described herein can include selecting and/or administering a treatment for kidney disease to a subject determined to have a score above a reference score, or a level of one or more of the variables evaluated herein above a reference level.

A number of treatments for kidney disease are known in the art. For example, standard treatments can include one or more of administration of medications to control blood pressure; e.g., angiotensin-converting enzyme inhibitors (ACEIs) or angiotensin II receptor blockers (ARBs), with a target blood pressure of less than 130/80 mm Hg; vitamin D supplementation, e.g., with synthetic analogs such as paricalcitol; treatment of hyperlipidemia, e.g., with statins; treatment of any hypothyroidism, e.g., with thyroid hormone replacement therapy (THRT) with L-thyroxine; controlling blood glucose levels (target hemoglobin A1c [HbA1C]<7%), e.g., with antidiabetic drugs or insulin; administration of renin-angiotensin system (RAS) blockers in subjects with diabetic kidney disease (DKD) and proteinuria; and administration of angiotensin-converting enzyme inhibitors (ACEIs) or angiotensin-receptor blockers (ARBs) in patients with proteinuria. In more advanced stages, other treatments can be added as needed, e.g., for anemia (erythropoiesis-stimulating agents, for example epoetin alfa or darbepoetin alfa); hyperphosphatemia (dietary phosphate binders and dietary phosphate restriction); hypocalcemia (Ca²⁺ supplements with or without calcitriol); volume overload (loop diuretics or ultrafiltration); metabolic acidosis (oral alkali supplementation); hyperparathyroidism (calcitriol, vitamin D analogues, or calcimimetics); and/or uremic manifestations (long-term renal replacement therapy (hemodialysis, peritoneal dialysis, or renal transplantation).

Alternatively or in addition, the methods can include selecting and/or administering a treatment directed at affecting suPAR-β3 integrin driven pathogenesis, e.g., an agent which inhibits soluble urokinase receptor (suPAR) activity and/or function and/or modulates expression soluble and/or membrane bound urokinase receptor (uPAR) and/or pathways associated with urokinase receptor, e.g., an antibody, aptamer, antisense oligonucleotide, a natural agent, or synthetic agent (see, e.g., WO 2010/054189), e.g., an α5β3 inhibitor, e.g., a monoclonal antibody that binds specifically to α5β3 and/or α5β5, e.g., VPI-2960B (Vascular Pharma, Research Triangle Park, N.C.) or CNTO95 as described in PCTUS2011/49563, or anti-CD61; a peptide comprising a RGD binding sequence, e.g., cylco-[Arg-Gly-Asp-D-Phe-Val], or a small molecule α5β3 inhibitor, e.g., a compound of the formula

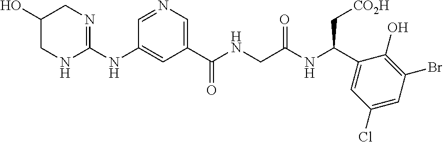 [SMILES: O=C(O)C[C@H](NC(=O)CNC(=O)c1cncc(NC2=NCC(O)CN2)c1)c1cc(Cl)cc(Br)c1O]

or a pharmaceutically acceptable salt thereof, or another compound as described in US2010/0297139; and/or ex vivo removal of suPAR from the subject's circulation, e.g., as described in WO2012/154218. Combinations of any of the above can also be administered.

### EXAMPLES

The invention is further described in the following examples, which do not limit the scope of the invention described in the claims.

### Example 1. Composite Diagnostic Assays Predict suPAR-β3 Integrin Driven Kidney Diseases

Reagents:

Level of human suPAR in serum was determined using commercially available ELISA assays from R&D. Level of suPAR in the urine was determined using identical ELISA.

Level of human IL6 in urine was determined using commercially available ELISA assay from Thermo Fisher Scientific (Life Technologies). AP5 antibody that recognizes active form of β3 integrin was from Blood Center of Wisconsin.

Methods:

AP5 Staining:

AP5 AB: Blood Center of Wisconsin; Paxillin: Abcam (Cat. #: a β32084). Human podocytes were proliferated at 33° C. and differentiated at 37 C for 10 days on coverslips in a 6-well plate. On day 10, the podocytes were serum starved overnight (RPMI 1640+anti-anti). Cells were then incubated with 10% healthy serum or patient sample for 24 h at 37 C. Healthy serum served as the negative control, and healthy serum supplemented with MnCl₂ at a final concentration of 625 μM served as the positive control. After treatment, samples were fixed with 4% paraformaldehyde for 20 minutes, washed and permeabilized with 0.3% Triton-X for 3 minutes, and blocked (5% donkey serum, 5% goat serum, in 1×PBS) for 45 minutes. The cells were incubated with the first primary antibody (anti-Paxillin, 1:300 Rabbit) for 1 hour. The secondary antibody goat-anti-rabbit 568 (1:2000, Invitrogen) was used. The cells were incubated with the second primary antibody (AP5, 1:50 Mouse) for 1 hour. The secondary antibody goat-anti-mouse 488 (1:1000, Invitrogen) was used. The coverslips were mounted with Flouroshield with DAPI (abcam, ab104139). Images were acquired using Zeiss LSM 5 Pascal. The detector gain, amplifier offset, and laser power settings were kept consistent for the collection of all images. The images were analyzed using ImageJ.

Additional Information for Image Analysis:

The files were opened in ImageJ and the channels were separated and images inverted. The threshold was set for each channel and kept consistent for the analysis of all images. For beta 3 integrin images, the nuclear staining was excised from each cell. Using the same freehand selection function, the cell was circled and the integrity density was measured for each channel. The data were exported into Microsoft Excel and the integrity density value of beta 3 integrin (AP5) staining of each cell was divided by the integrity density value of focal adhesion (paxillin) staining of each cell, and normalized to the negative control.

Detection of Low Molecular Weight Proteins Using IP and Western Blot Analysis:

Serum samples were diluted 1:1 with RIPA buffer (Pierce RIPA Buffer, Product number 89901) containing protease inhibitor cocktail tablet (Roche, Product number 11836170001). Streptavidin Mag Sepharose beads (GE Healthcare, Product code: 28-9857-99) were rinsed twice with RIPA buffer containing protease inhibitor and added to the serum samples (beads:serum samples=1:20) and incubated for 1 hour at room temperature on a tube rotator RotoFlex (Argos, Catalog number R2000). uPAR (R4)-BSA Free (Novusbio Product number NBP2-41379-0.2 mg) or ATN 615 antibody was biotinylated using EZ-link Micro Sulfo-NHS-Biotinylation Kit (ThermoFisher Scientific, Catalog number: 21925) and stored at 4° C. until use. To the precleared serum samples biotinylated uPAR (R4)-BSA Free or ATN615 antibody was added (antibody:dilute precleared serum=1:530) and incubated on a tube rotator for 1 h at room temperature. Washed Streptavidin Mag Sepharose beads (beads:serum samples=1:20) were then added to the samples and incubated for another hour on the tube rotator at room temperature. The unbound protein fractions were removed and the beads were rinsed once and then washed for 10 min with RIPA buffer on the tube rotator. To the bound fraction 25.5 μL N-Glycanase reaction buffer (working solution) and 2.5 μL of denaturation solution (PROzyme, Code: WS0012) were added and the samples were heated at 70° C. for 3 min. The samples were then cooled on ice for 1 min. Detergent solution (2.5 μL) (PROzyme, Code: WS0013) and 2-4 μL N-Glycanase (working solution) were then added to the samples and incubated overnight at 37° C. Working solution of N-Glycanase reaction buffer was prepared by diluting the buffer stock (PROzyme, Code: WS0010) 5× with DI water. Working stock of N-Glycanase was prepared by diluting the enzyme stock (PROzyme, Code:GKE-5006A) 10× with the working solution of N-Glycanase reaction buffer.

The proteins were eluded from the beads by boiling the samples in Laemmili Sample Buffer (Biorad, catalog number 161-0747) supplemented with additional SDS solution and 2-marcaptoethanol. The samples were then subjected to SDS-PAGE analysis using 4-20% Mini-PROTEAN TGX gels (Biorad, Catalog number 456-1094). The proteins were then transferred to a PVDF membrane and subjected to Western blot analysis using Rabbit anti-UPAR (Bethyl, Product number A304-462A) (1:1250) or Anti-PLAUR antibody produced in rabbit (Sigma-Aldrich, Product number HPA050843-100 uL) (1:1000), followed by goat anti-rabbit-HRP (1:3333).

### Example 1.1: suPAR Levels in Plasma do not Correlate with β3 Integrin Activation

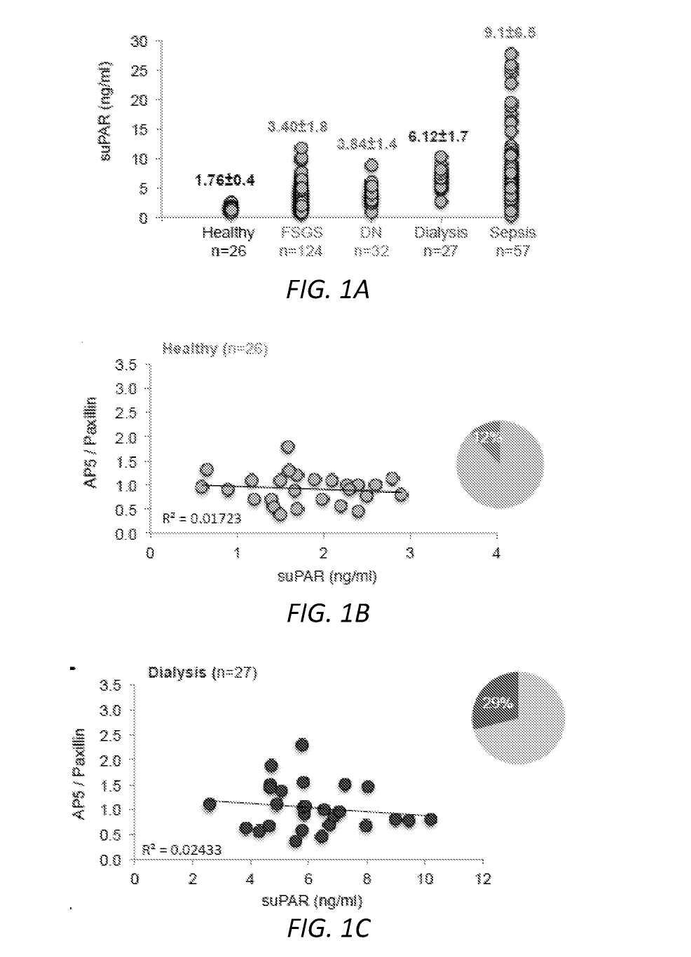

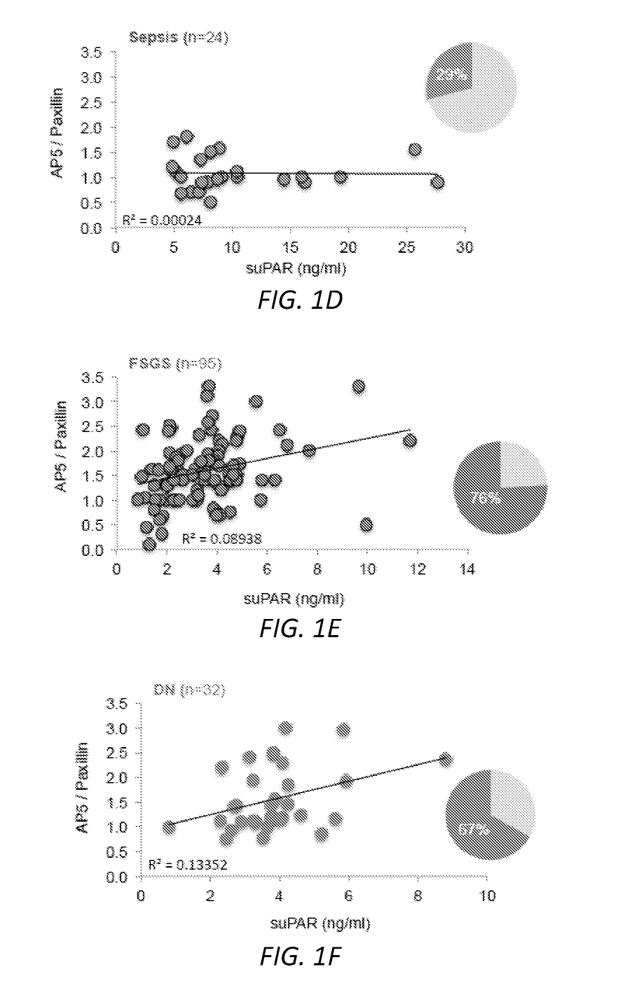

It has been suggested that suPAR drives podocyte injury by activating β3 integrin on podocytes (5). Thus, we examined correlation between suPAR concentrations and β3 integrin activation (FIG. 1). As seen before, suPAR levels in the plasma were elevated upon multiple pathogenic conditions, such as focal segmental glomerulosclerosis (FSGS), diabetic nephropathy (DN), in patients on dialysis, or in sepsis (FIG. 1A).

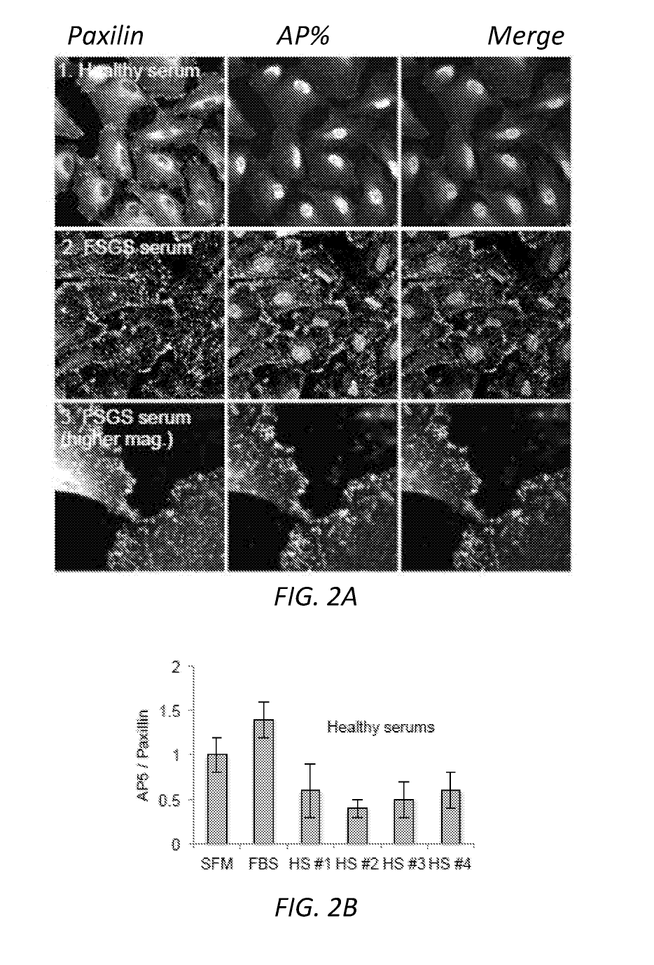

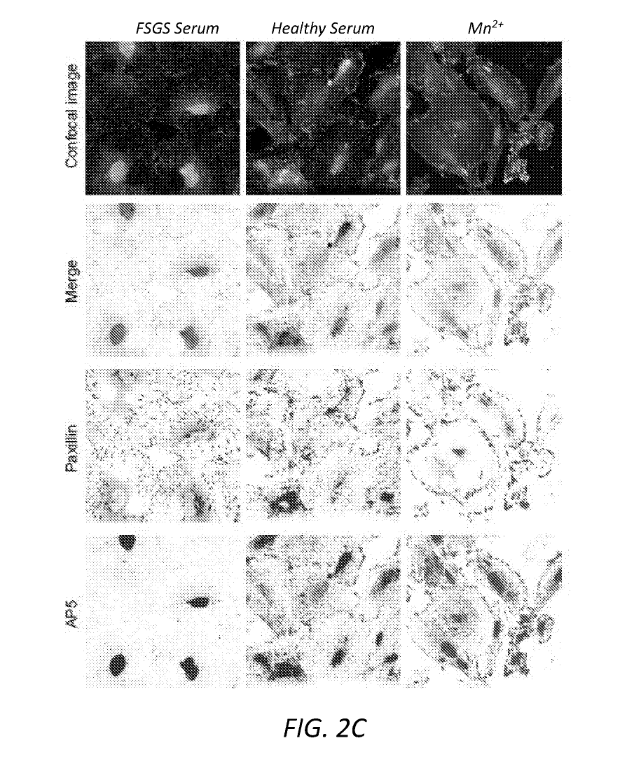

Since the original published assay that detected β3 integrin activation in human cultured podocytes lacked a quantifiable component, and in order to compare β3 integrin activation induced by different serums in a most relevant and quantifiable manner, we modified the original assay (FIG. 2) so that it measured the ratio of activated integrin level (represented by AP5 staining in FIG. 2A) over total amount of focal adhesions (FAs, determined by Paxillin staining in green in FIG. 2A). No significant difference in the level of β3 integrin activation between different healthy serums (HS), or that compared to the addition of 10% fetal bovine serum (FBS) standardly used to grow podocytes in culture was detected (FIG. 2B). In order to compare data sets performed on different days, the levels of β₃ integrin activation in the presence of different sera were compared to that of a healthy serum, which was used as a standard in each experiment. The signal was quantified using ImageJ (see also Methods), and representative data set are show in FIG. 2C. Ratio between AP5 staining and paxillin for healthy serum was adjusted to 1, and all other ratios are calculated with respect to the healthy serum in each experiment. Addition of Mn²⁺ is used to non-selectively activate all integrins on the surface of podocytes including β3 integrin, thus determining the maximal level of β3 integrin activation in the assay (FIG. 2C). Ratios between AP5 signal and paxillin that were >1.2 were considered a positive signal (presence of β3 integrin activation).

As shown in FIG. 1, only ˜12% of serum samples from healthy individuals exhibited β3 integrin activation (FIG. 1B), and that number was increased to ˜25% for serums from patients on dialysis or in sepsis (FIGS. 1C and 1D). Importantly, β3 integrin activation did not correlate with suPAR concentrations. Indeed, extremely high levels of suPAR detected in serums of patients in sepsis (10-30 ng/ml) did not necessarily lead to β3 integrin activation (FIG. 1D). Interestingly, the majority of serum samples from patients suffering from FSGS (˜76%) exhibited β3 integrin activation. Even more surprising was the fact that high percent of serums (˜67%) from patients with DN also exhibited β3 integrin activation, suggesting that in some instances DN pathology might also be driven by suPAR-β3 pathway.

### Example 1.2: β3 Integrin Activation by Human Serums is in Part suPAR Dependent

Despite the fact that β3 integrin activation was originally linked to elevated levels of suPAR in FSGS subjects (5), lack of correlation between suPAR levels and β3 integrin activation suggested three distinct possibilities. First, β3 integrin activation by human serums was not suPAR-dependent. Second, β3 integrin activation was in part suPAR-dependent but it required a yet unidentified modifiable factor. Third, suPAR per se might be distinctly modified.

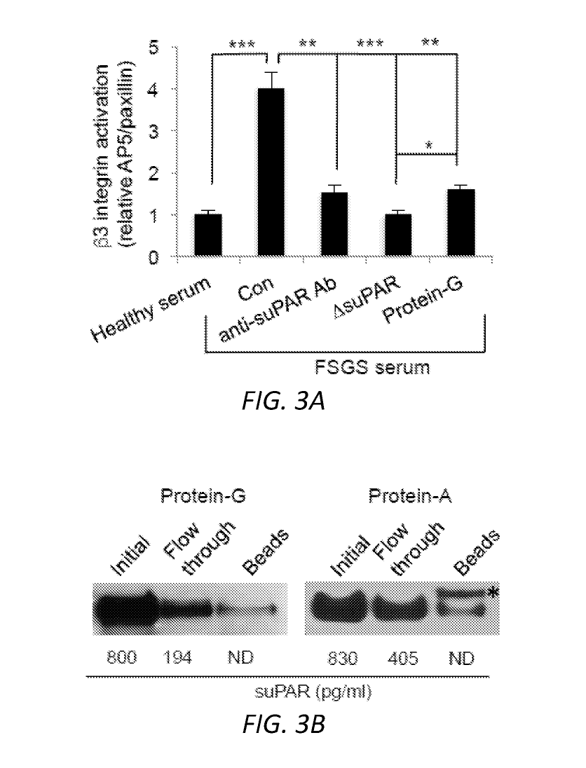

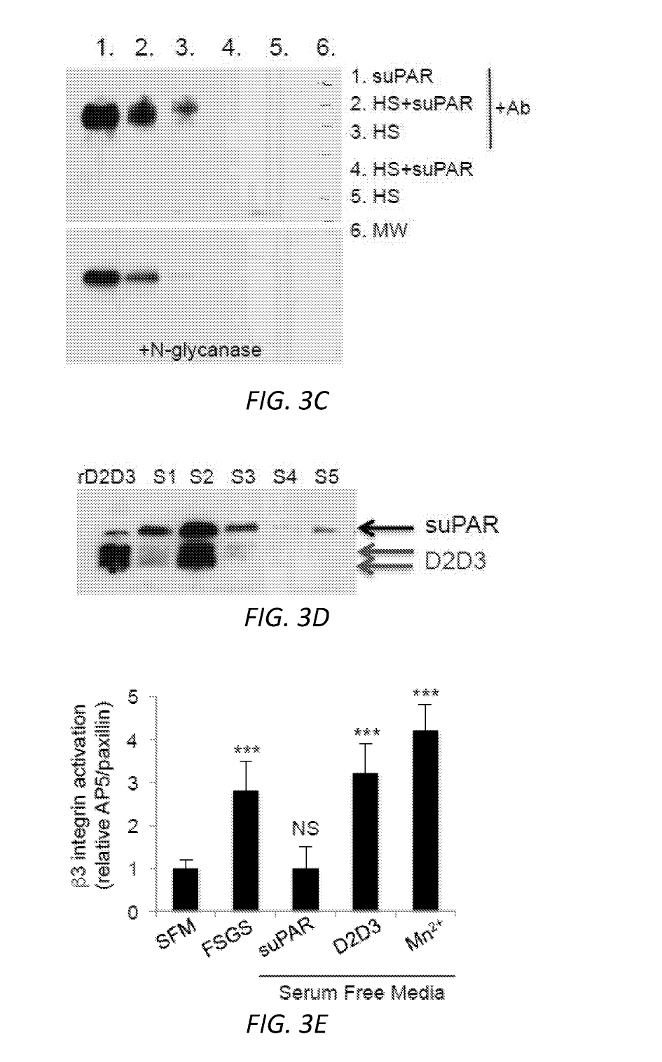

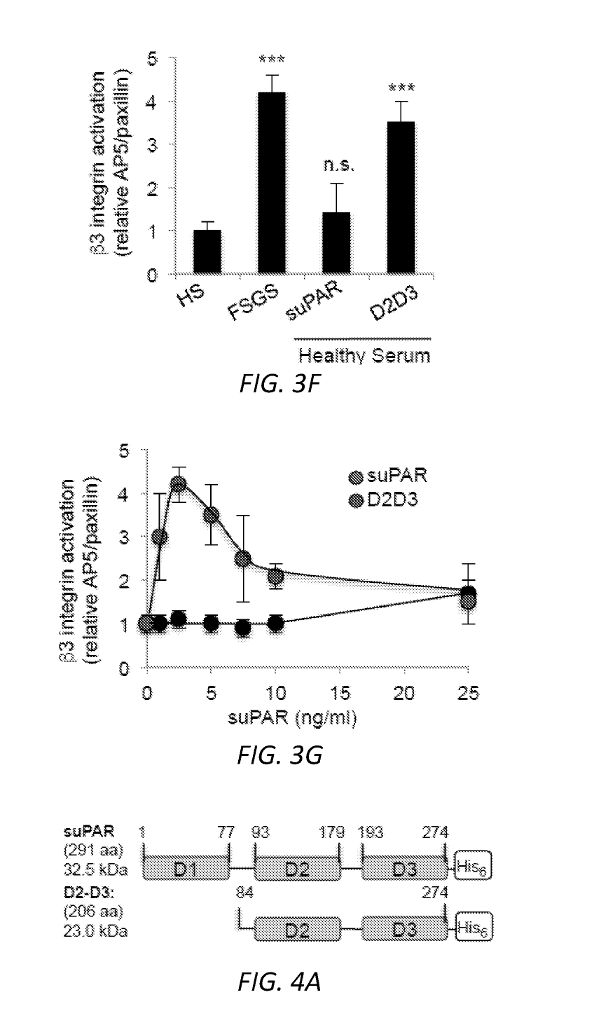

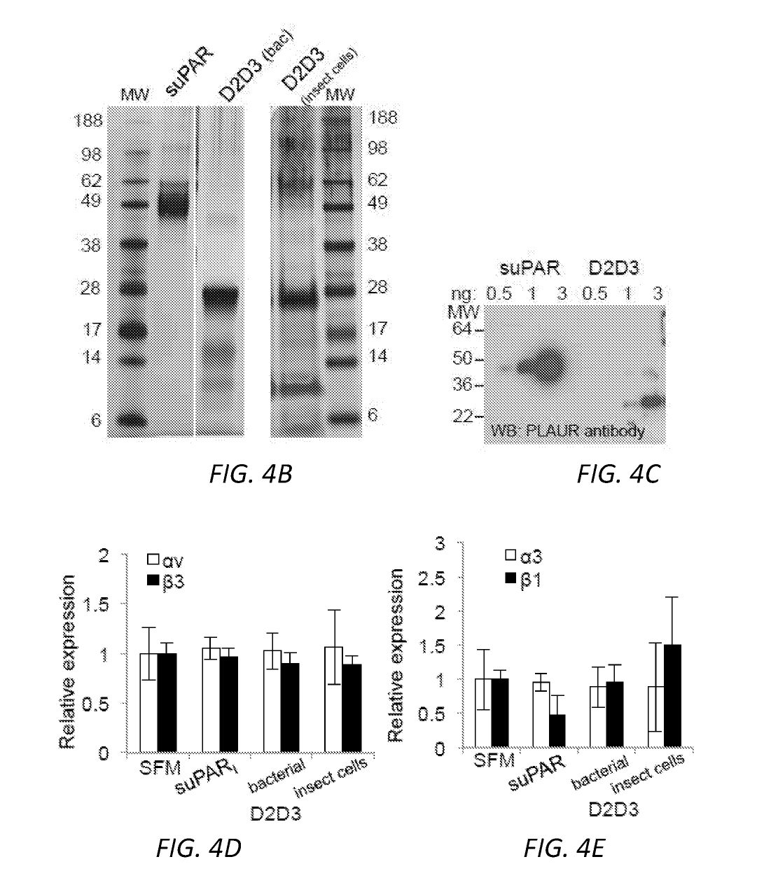

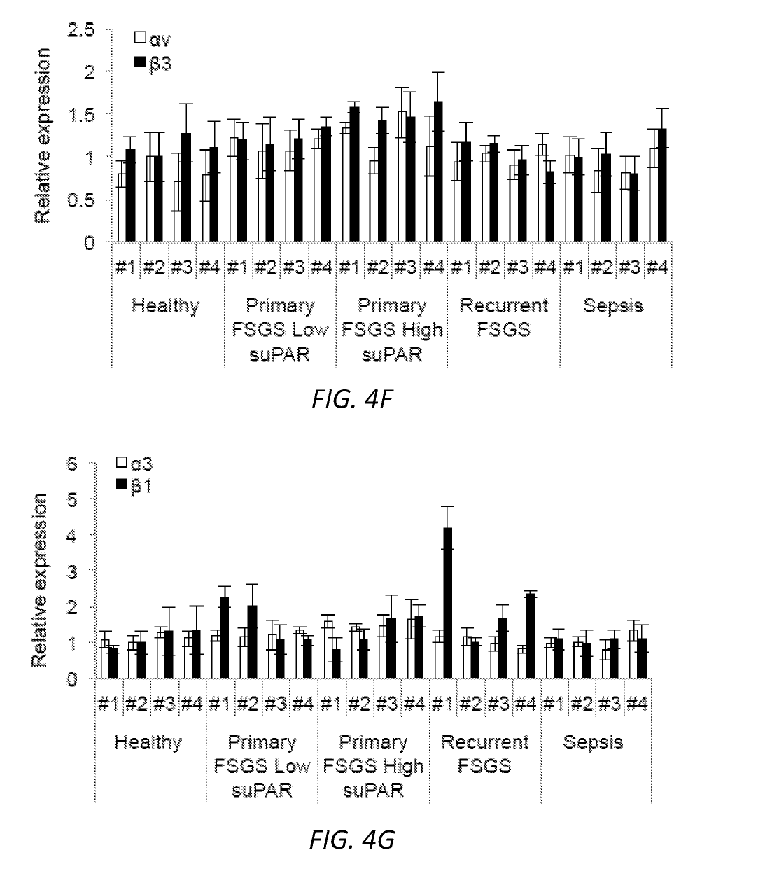

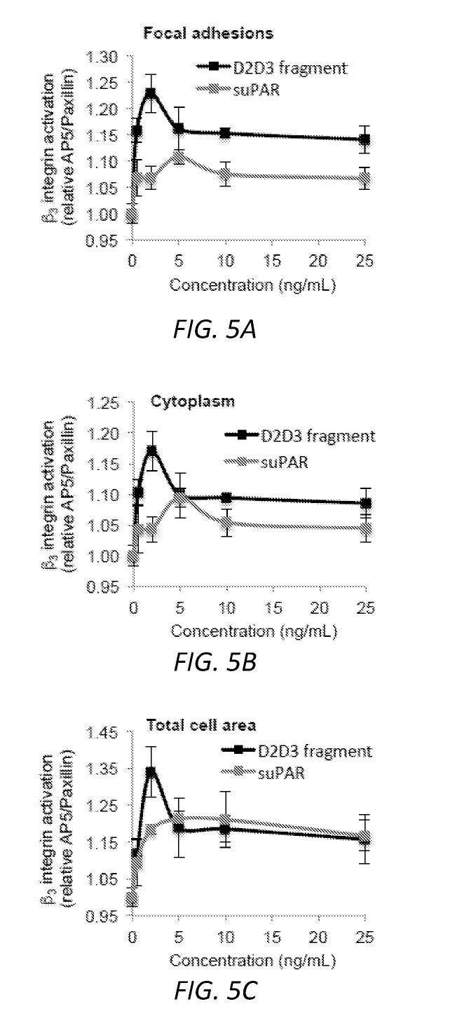

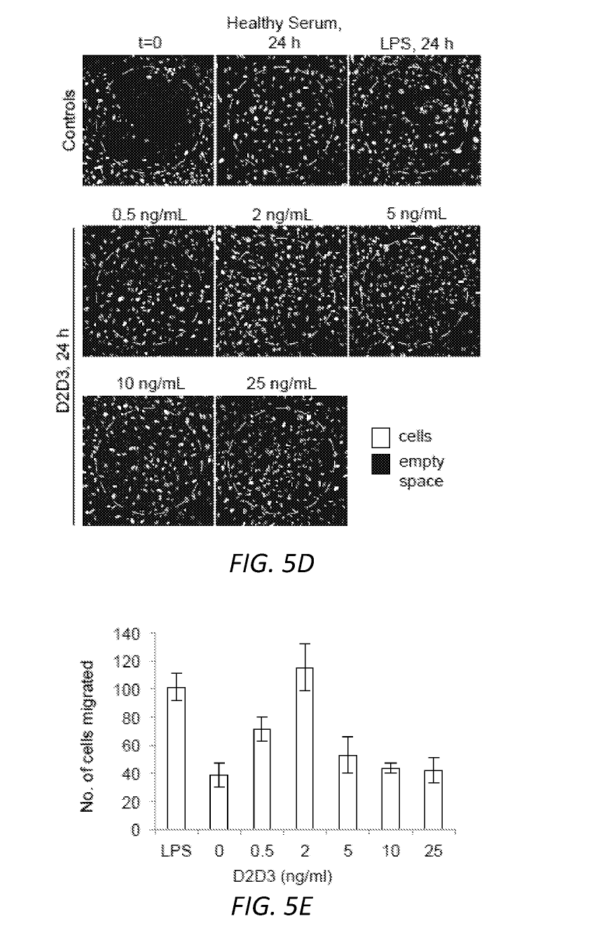

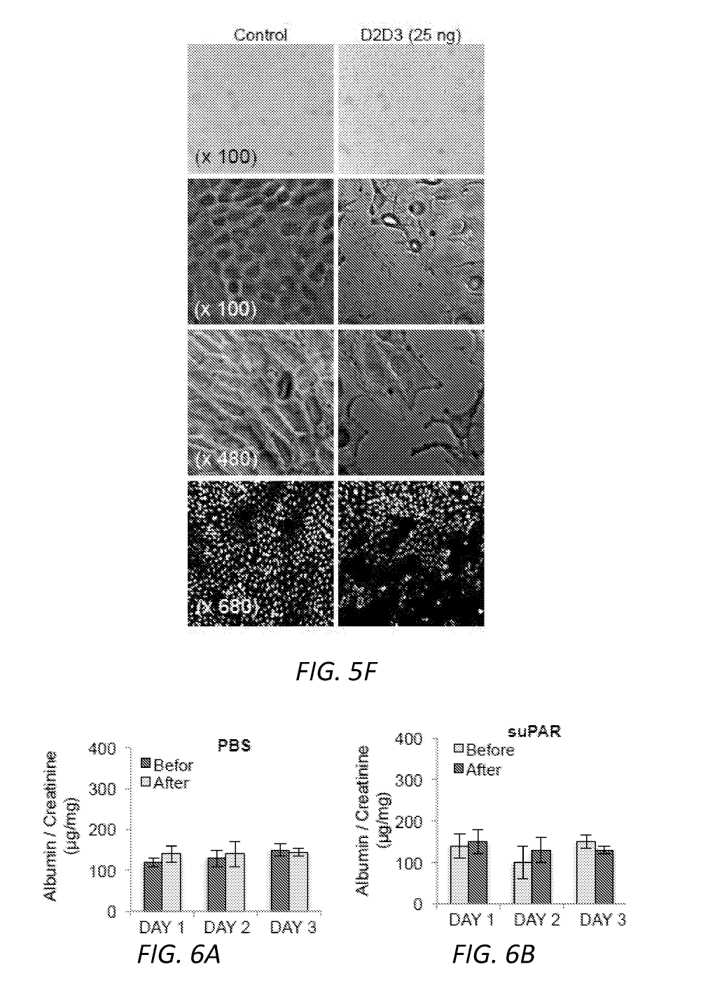

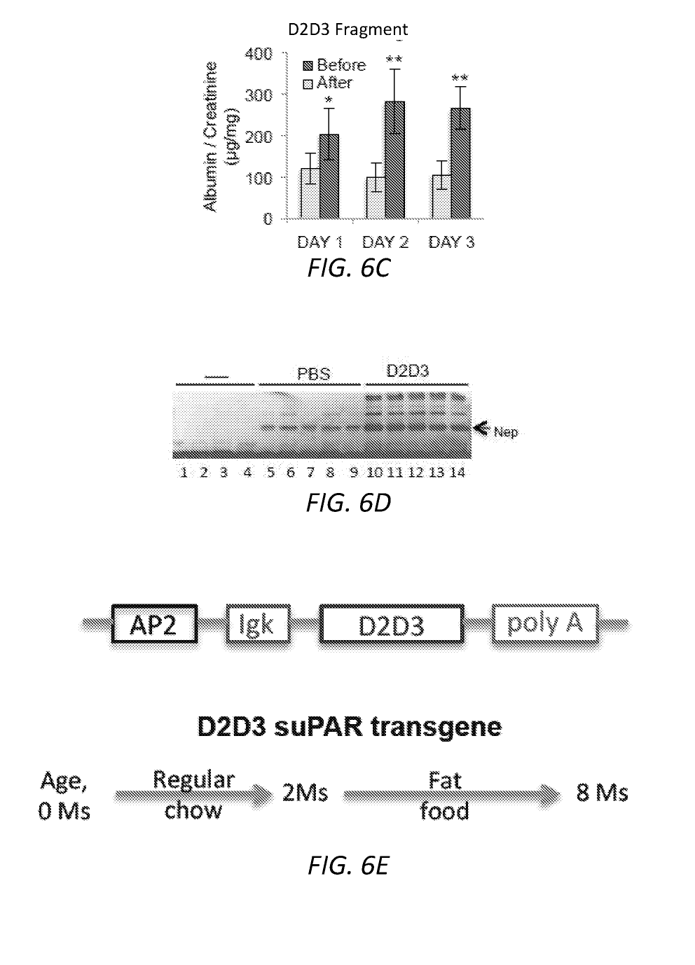

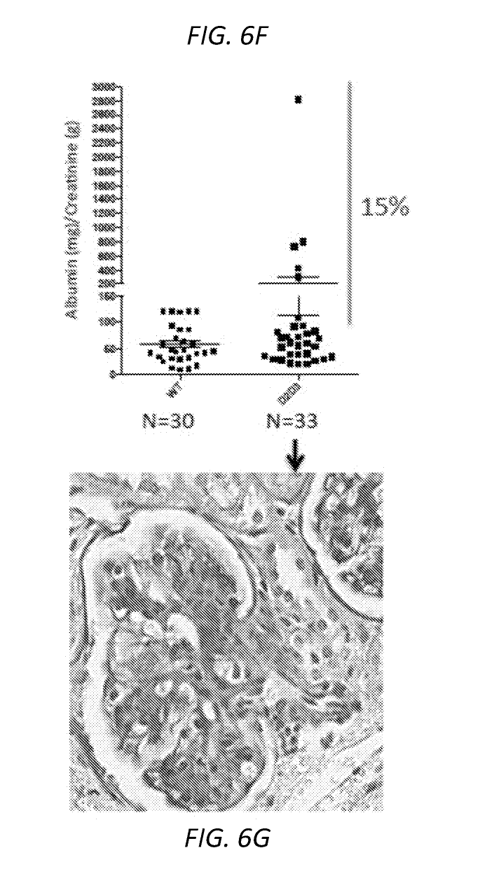

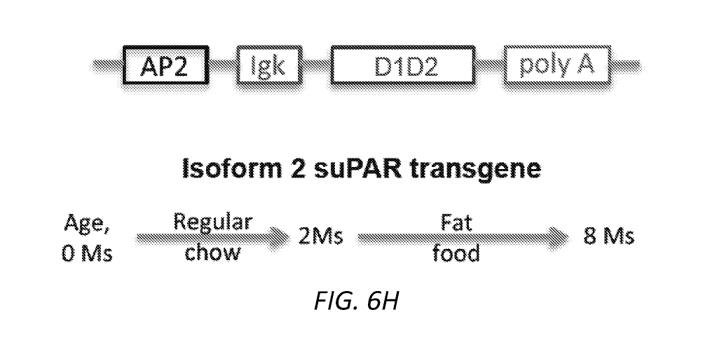

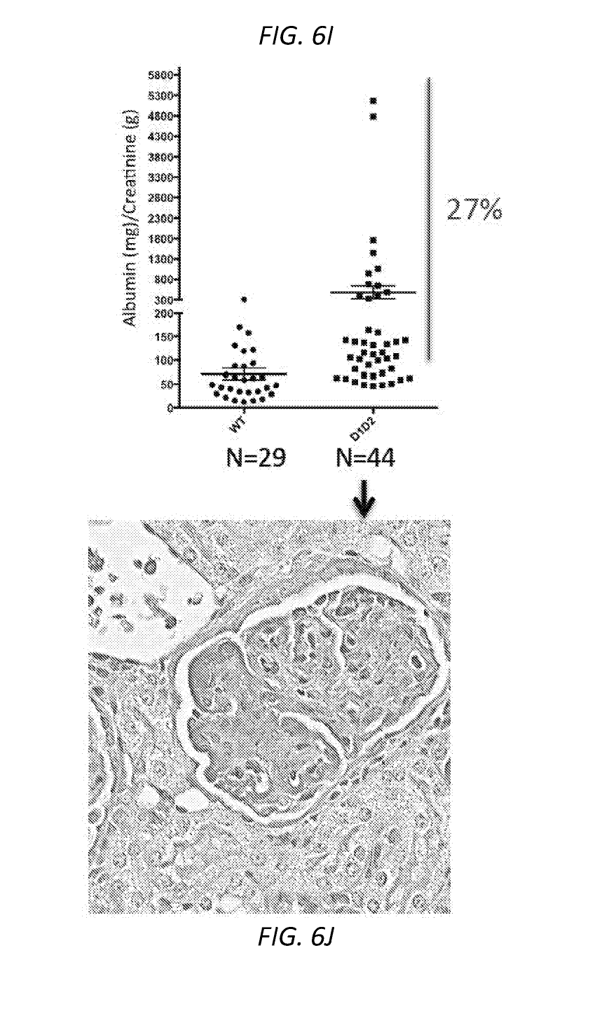

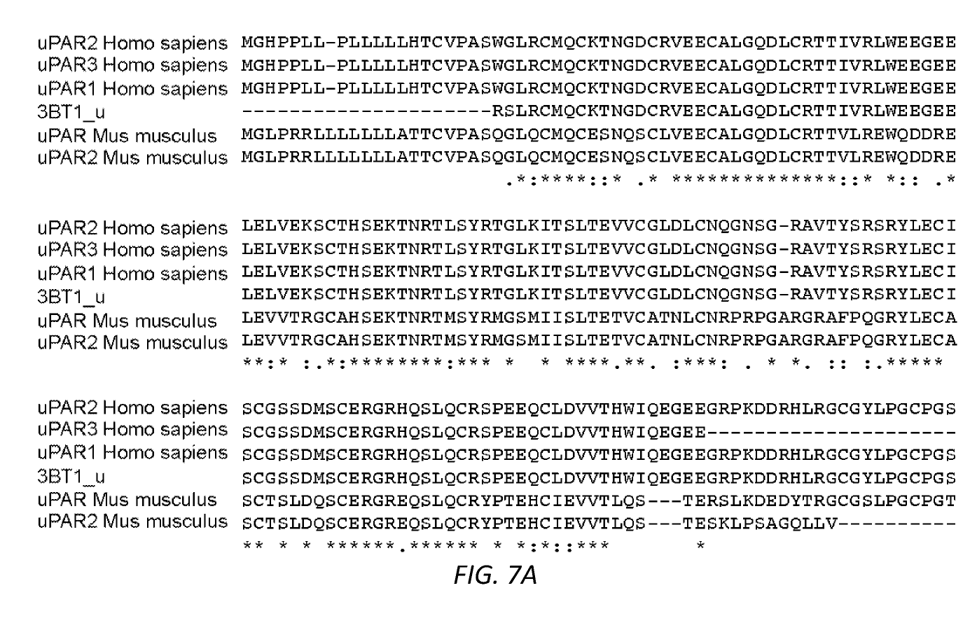

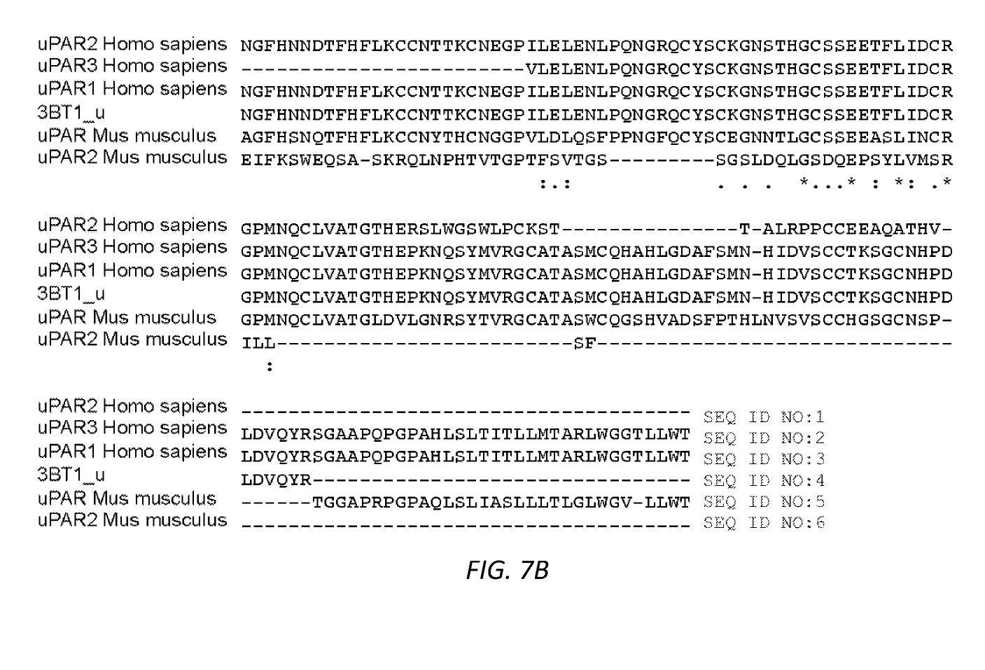

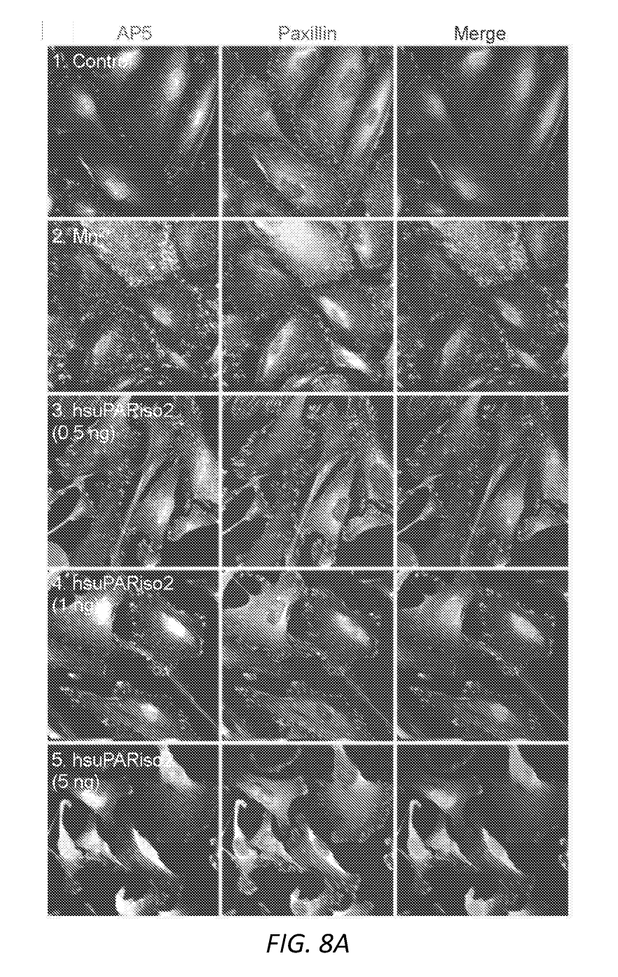

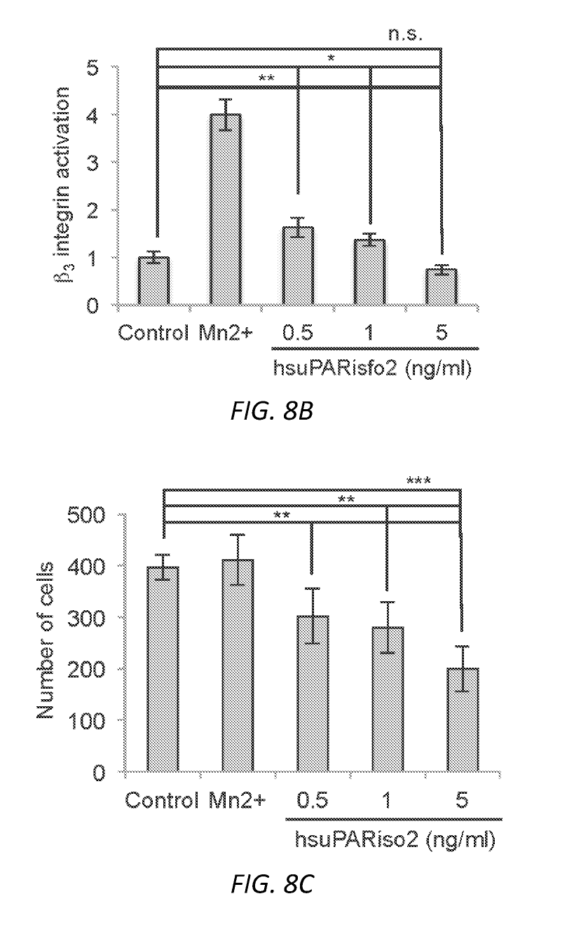

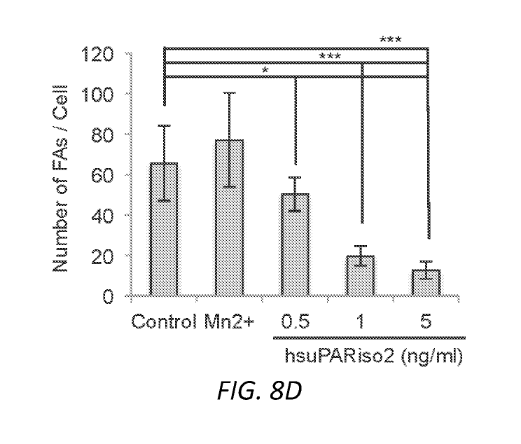

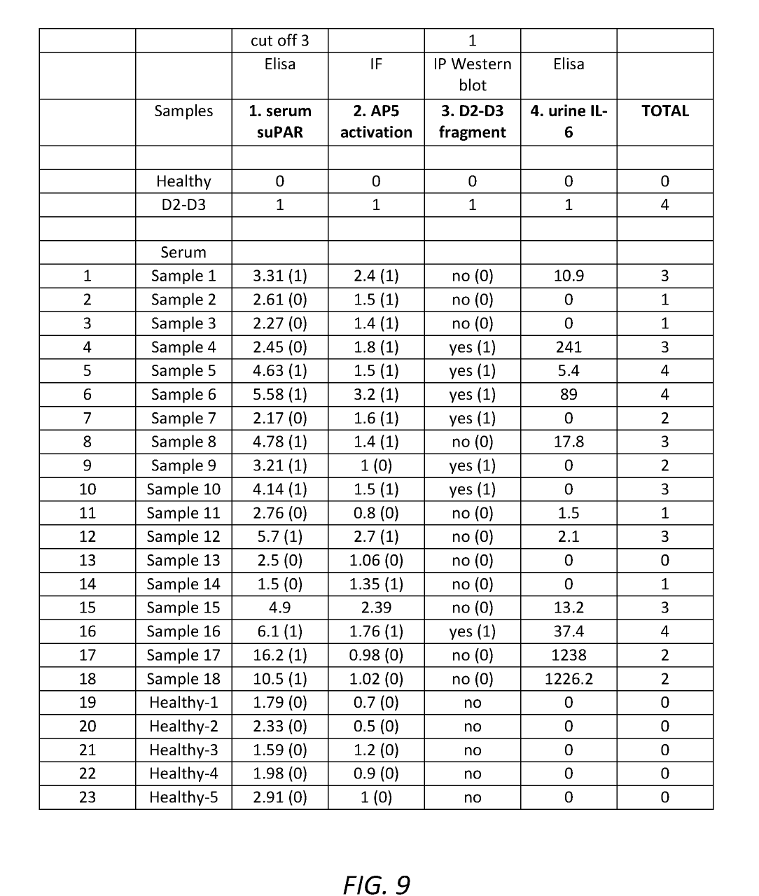

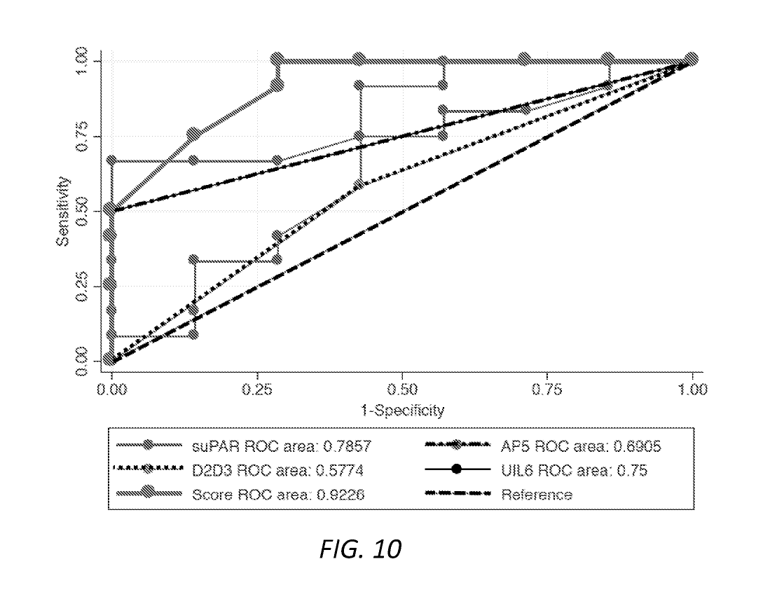

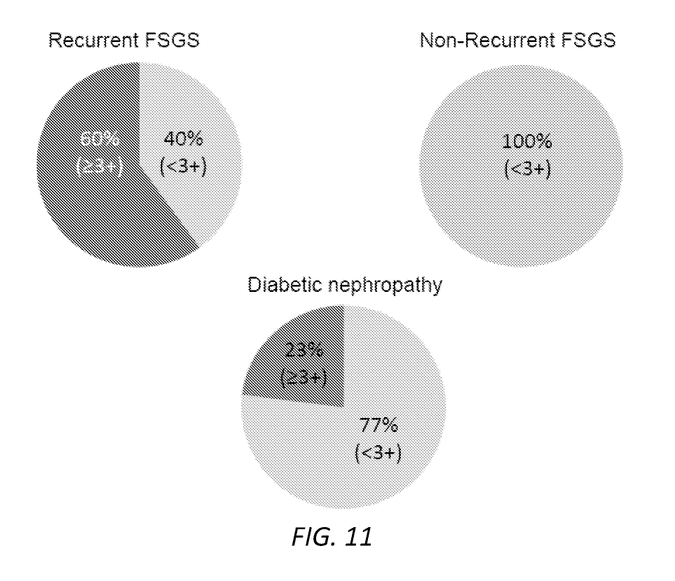

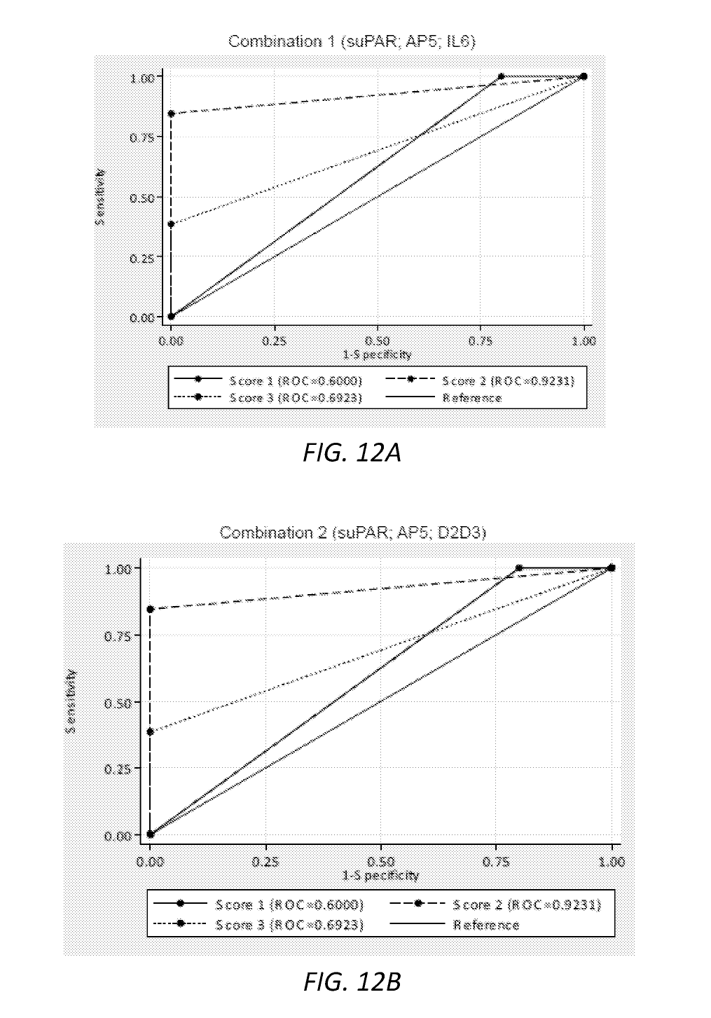

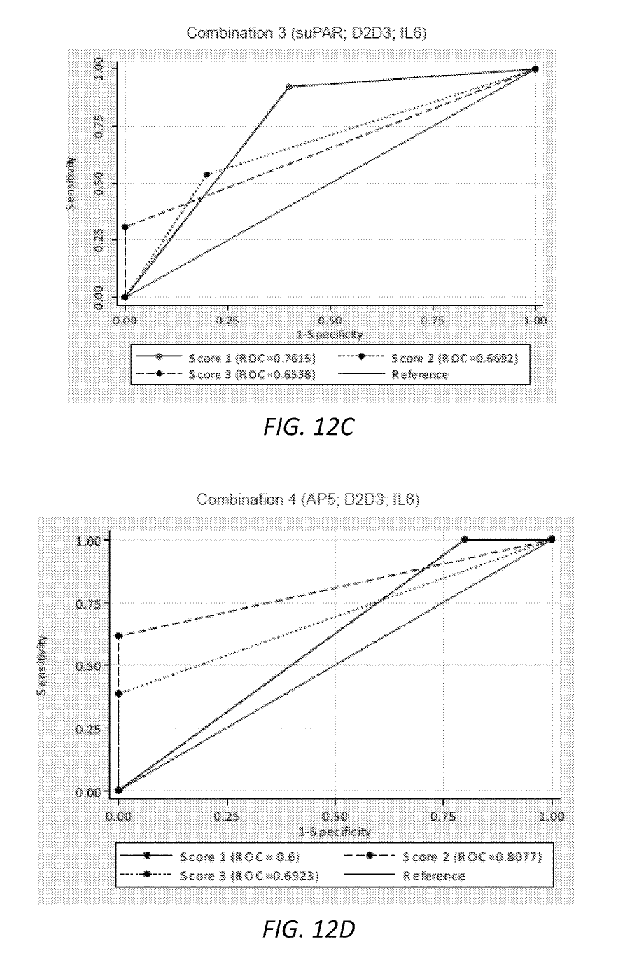

We first attempted to test whether observed β3 integrin activation was suPAR-dependent. To this end, FSGS serum was incubated with anti-suPAR antibody to test whether this procedure could block integrin activation. As shown in FIG. 3A, addition of anti-suPAR antibody significantly diminished ability of FSGS serum to activate β3 integrin. In addition, removal of suPAR using anti-suPAR antibody bound to Protein G beads (immunoprecipitation procedure) also significantly lowered the ability of FSGS serum to activate β3 integrin (FIG. 3A, ΔsuPAR column). Ability of Protein G beads that are not conjugated to anti-suPAR antibody to also significantly lower ability of FSGS serum to activate β3 integrin (FIG. 3A, Protein G column) was due to propensity of suPAR to non-specifically bind Protein-G and Protein—A beads (FIG. 3B). Together, those data demonstrated that detected β3 integrin activation was indeed suPAR-dependent.

We next examined whether there was a difference in the form(s) of suPAR present in FSGS serums that activate β3 integrin versus those present in healthy serums. While uPAR is encoded by one gene, at least 3 different splice variants have been identified so far (FIG. 7). In addition, uPAR consists of three domains (D1, D2, D3) and can be cleaved between D1 and D2 domain to generate two fragments: D1 and D2D3 fragment (9). D2D3 fragments have been shown to promote cell motility (10) and to bind β3 integrin. In addition, it has been show that mouse variant 2 (FIG. 7) when expressed in mouse causes proteinuria and glomerular injury similar to FSGS (5). Thus, we next attempted to examine suPAR status in serum. Since suPAR is present at very low concentrations (healthy levels are <3 ng/ml), in order to detect suPAR in serum we immunoprecipiated (IP) suPAR using anti-suPAR antibody, and examined the precipitated proteins using Western blot analysis (FIG. 3C). To confirm specificity of suPAR using this procedure, recombinant human suPAR (FIG. 3C, lane 1) was added to human serum of suPAR (FIG. 3C, lane 2). Given the fact that suPAR is also contains 5 glycosylation sites, samples were de-glycosylated using N-glycanase. As shown in FIG. 3, this procedure efficiently and specifically IP-ed suPAR from the human serum (FIG. 3C, lane 4), but only in the presence of anti-suPAR antibody (FIG. 3C, lanes 5, 6). When multiple serums were tested by this procedure, lower molecular weight proteins were detected (FIG. 3D, lane S2 and red arrows). Those lower molecular weight proteins migrated with the speed of D2D3 fragment generated by cleaving recombinant human suPAR using chymotrypsin, suggesting presence of D2D3 fragment in serums on some of the patients.

In order to test whether D2D3 fragment had ability to activate β3 integrin by itself, we expressed and purified human D2D3 fragment (schematic diagram shown in FIG. 4A) in insect cells and bacteria. The purity of the proteins is shown in FIG. 4B. Both proteins, full length suPAR and D2D3 fragment were recognized by PLAUR antibody, though D2D3 fragment to a lesser extent, suggesting that levels of fragment detected in human serum might be underestimated. Importantly, addition of D2D3 fragment potently activated β3 integrin on human podocytes (FIG. 3E). Since activation was present using serum free media, this data demonstrate that D2D3 fragment does not require an additional serum modifier to potently activate β3 integrin. In addition, at exact same physiological concentration (2 ng/ml) full-length suPAR did not induce significant activation (FIG. 3E). Addition of D2D3 fragment to healthy serum transformed the serum from non-activating (HS bar graph in FIG. 3F) to activating. Of note, addition of suPAR or D2D3 fragment did not alter expression levels of αVβ3 and α3β1 (FIG. 4D,E), further suggesting that observed β3 integrin activation was indeed due to conformational switch within β3 integrin and not due to overall increase in β3 integrin levels in the cell. Consistent with these experiments, comparison between activating (recurrent FSGS serum), non-activating serum and healthy serums did not detect significant alterations in expression levels of αVβ3 and α3β1 integrins in human podocytes (FIGS. 4F,G) further demonstrating specific effects of activating serums on the conformational switch within β3 integrin, and not its levels.

Concentration dependence of β3 integrin activation with regard to D2D3 exhibited cooperative behavior (small changes in the concentration had significant consequences on β3 integrin activation) (FIG. 3G). The peak of activation was observed at ˜2 ng/ml, with higher concentrations leading to lover activation, most likely due to so called “hyper activation” that can result in integrin internalization (ref). Identical activating profile was observed using high throughput assays (FIG. 5A-C). In addition, D2D3-induced integrin activation increased podocyte motility (FIG. 5 D, E). Importantly, the concentration of D2D3 fragment (2 ng/ml) that was associated with the highest level of β3 integrin activation also resulted in the greatest motility. Increase in D2D3 concentrations (5-25 ng/ml) was associate with lover cell motility and indeed cell detachment (FIG. 5F), most likely due to internalization of β3 integrin due to hyper-activation. In summary, our data suggest that presence of D2D3 fragment in the serum might underlie ability of that serum to induce β3 integrin activation.

### Example 1.3: D2D3 Fragment Induces Podocyte Damage and Proteinuria in Mice

Ability of D2D3 fragment to induce potent β3 integrin activation and motility suggested that D2D3 might cause podocyte injury leading to proteinuria when present in circulation. Thus, we next injected recombinant proteins into the tail vain of mice. As shown in FIGS. 6A and 6B, animals injected with PBS (vehicle control) or suPAR did not exhibit proteinuria (determined based on Albumin/creatinine ratio in FIG. 6). In contrast, injection of D2D3 resulted in transient proteinuria and lead to detectable presence of nephrin in the urine of proteinuric animals. Since nephrin is a transmembrane protein specifically present in podocytes, and since it has been shown that podocyte injury often leads to release of nephrin from the podocytes together, those data show that D2D3 in circulation can induce podocyte injury.

To further examine the ability of D2D3 fragment to induce podocyte injury we generated D2D3-trangenic mice expressing D2D3 form adipocytes (FIG. 6E). The protein expression was induced by putting the animals onto the fat diet at 2 months of age (FIG. 6F). While a number of animals exhibited microalbuminuria, approximately 15% of animals developed significant proteinuria and their glomerulus showed signs of injury such as moderate mesangial expansion (FIG. 6G). Together, these data show that D2D3 can cause podocyte injury when present in the circulation.

### Example 1.4: suPAR Isoform 2 Causes Proteinuria and Glomerular Injury

Originally, it was shown that expression of mouse splice variant 2 causes FSGS type of glomerular injury in mice(5) and FIG. 7. Those experiments were performed by electroporating DNA encoding mouse isoform 2 protein in mice (5). Consistent with those original observations, constitutive expression of isoform 2 in mouse form adipocytes (FIG. 6H) resulted in ˜27% of animals exhibiting signs of proteinuria (FIG. 6I) and glomerular injury (FIG. 6J). Together those data suggested that while presence of D2D3 in circulation can induce podocyte injury, expression of isoform 2 can do the same. Indeed, while addition of human suPAR isoform 2 induced moderate β3 integrin activation at sub physiological concentration (FIGS. 8A and 8B, 0.5 ng/ml), isoform 2 induced potent cell detachment (FIG. 8C) due to loss of focal adhesions (FA in FIG. 8D). It is worth nothing that isoform 2 lacks part of the domain 3 as well as GPI-anchor sequence (FIG. 7) thus it is expected to be directly secreted into the circulation and to exhibit lower molecular weight then the full length protein. The last observation is important given the presence of lower molecular weight proteins in human serums by Western blot analysis. Together, our data suggest that suPAR in circulation have multiple ways of activating β3 integrin, and thus inducing podocyte injury: via formation of D2D3 fragment, and/or expression of distinct splice variant such as isoform 2.

### Example 1.5: Establishment of Composite Score that Identifies suPAR-β3 Integrin in Podocyte Injury

Based on our studies, it seemed reasonable to suggest that glomerular injury in subset of patients suffering from FSGS might be driven by suPAR-β3 integrin pathway. In addition, given ability of some of DN serums to activate β3 integrin (FIG. 1F), data suggested that this pathway might also underlie some other types of CKD such as DN. To explore this idea further we established a composite score assay that can efficiently identify suPAR-β3 integrin pathogenic pathway in a non-invasive way. Since this pathway has been implicated specifically in recurrent FSGS we decided to test ability of several assays/biomarkers to efficiently distinguish recurrent from non-recurrent FSGS. In addition, recurrent vs non-recurrent FSGS represented highly uniform patient population since all patients were diagnosed using kidney biopsy, they all went through ESRD (end stage renal disease) and dialysis, they all got new kidney and are on similar immunosuppression therapies. What distinguished them is that in certain instances diseases recurred and in some it did not. Thus, we measured suPAR levels in their serums and urine, we determined the ability of serums to activate β3 integrin, we determined whether their serums contain lower molecular weight proteins using Western blot analysis and we also measured level of IL6 in their urine.

Samples from patients with recurrent FSGS (19 samples) were compared to samples from patients with non-recurrent FSGS (7 samples) using a weighted average formula to generate a composite score assay.

The composite score was calculated based on the following algorithm:

score=0.253×(serum suPAR)+0.282×(β3 integrin activation)+0.212×(D2D3fragment)+0.253×(urine IL-6)

A value of 0 or 1 was assigned to each of the four parameters as described above. In the present cohort, a score of >0.5 was considered positive for suPAR-β3 integrin driven podocyte injury.

Representative scoring is shown in FIG. 9. As shown in FIG. 10, ROC (receiver operating characteristic) analysis showed that while each parameter exhibited significant ability to separate non-recurrent from recurrent FSGS, the joined score of 0.922 was impressive when all four parameters were determined. Presence of 3 positive parameters was detected in 60% of subject with recurrent FSGS and not in the single subject with non-recurrent FSGS. Interestingly, ˜23% DN subjects exhibited ≥3+ further suggesting that the suPAR-β3 integrin pathway might underlie renal pathology in a subset of patients suffering from DN (FIG. 11). Together, these data establish the composite score assay as a viable tool to identify suPAR-β3 integrin pathway that underlies podocyte injury in CKD.

FIGS. 12A-D show ROC (receiver operating characteristic) curves with single or combinations of 2 or 3 different parameters. Area under the curve was calculated for each one of the 3 indicated parameters (Score 1); combination of any two parameters (Score 3) and finally the combination of three parameters (Score 3). FIG. 12A is suPAR, AP5, and IL6 parameters. FIG. 12B is suPAR, AP5, and low molecular weight suPAR parameters. FIG. 12C is suPAR, low molecular weight suPAR, and IL6 parameters. FIG. 12D is AP5, low molecular weight suPAR, and IL6 parameters. Data show that any given parameter exhibited statistically significant value (area under the curve of ROC was between 0.6 and 0.76 in all combinations). While the combination of any two given parameters increased statistical significance (Score 2 was between 0.92 to 0.669) by increasing sensitivity, it also resulted in drop of specificity. This means that while any 2 given values could separate healthy from patients that got a kidney transplant (FSGS subjects), this would not be sufficient to predict that those FSGS subject exhibit suPAR-b3 integrin pathogenic pathway. Indeed, inclusion of the third parameter in all combinations decreased ROC values (0.65-0.69) due to drop in both sensitivity and specificity. Only when all 4 parameters were included, as in FIG. 10, did ROC value become 0.922.

### REFERENCES

1. Shih N Y, Li J, Karpitskii V, Nguyen A, Dustin M L, Kanagawa O, et al. Congenital nephrotic syndrome in mice lacking CD2-associated protein. Science. 1999; 286(5438):312-5.
2. Kaplan J M, Kim S H, North K N, Rennke H, Correia L A, Tong H Q, et al. Mutations in ACTN4, encoding alpha-actinin-4, cause familial focal segmental glomerulosclerosis. Nature genetics. 2000; 24(3):251-6.
3. Santin S, Garcia-Maset R, Ruiz P, Gimenez I, Zamora I, Pena A, et al. Nephrin mutations cause childhood- and adult-onset focal segmental glomerulosclerosis. Kidney international. 2009; 76(12):1268-76.
4. Brown E J, Schlondorff J S, Becker D J, Tsukaguchi H, Tonna S J, Uscinski A L, et al. Mutations in the formin gene INF2 cause focal segmental glomerulosclerosis. Nature genetics. 2010; 42(1):72-6.
5. Wei C, El Hindi S, Li J, Fornoni A, Goes N, Sageshima J, et al. Circulating urokinase receptor as a cause of focal segmental glomerulosclerosis. Nature medicine. 2011; 17(8):952-60.
6. Hayek S S, Sever S, Ko Y A, Trachtman H, Awad M, Wadhwani S, et al. Soluble Urokinase Receptor and Chronic Kidney Disease. The New England journal of medicine. 2015; 373(20):1916-25.
7. Theilade S, Lyngbaek S, Hansen T W, Eugen-Olsen J, Fenger M, Rossing P, et al. Soluble urokinase plasminogen activator receptor levels are elevated and associated with complications in patients with type 1 diabetes. Journal of internal medicine. 2015; 277(3):362-71. Epub 2014 Jun. 23.)
8. Huang J, Liu G, Zhang Y M, Cui Z, Wang F, Liu X J, et al. Plasma soluble urokinase receptor levels are increased but do not distinguish primary from secondary focal segmental glomerulosclerosis. Kidney international. 2013; 84(2):366-72.
9. Smith H W, Marshall C J. Regulation of cell signalling by uPAR. Nature reviews Molecular cell biology. 2010; 11(1):23-36.
10. Kjoller L. The urokinase plasminogen activator receptor in the regulation of the actin cytoskeleton and cell motility. Biological chemistry. 2002; 383(1):5-19.
11. Haraldsson B, Nystrom J, Deen W M. Properties of the glomerular barrier and mechanisms of proteinuria. Physiological reviews. 2008; 88(2):451-87.
12. Kerjaschki D. Caught flat-footed: podocyte damage and the molecular bases of focal glomerulosclerosis. The Journal of clinical investigation. 2001; 108(11):1583-7.
13. Reiser J, von Gersdorff G, Simons M, Schwarz K, Faul C, Giardino L, et al. Novel concepts in understanding and management of glomerular proteinuria. Nephrology, dialysis, transplantation: official publication of the European Dialysis and Transplant Association—European Renal Association. 2002; 17(6):951-5.
14. Reiser J, Sever S. Podocyte biology and pathogenesis of kidney disease. Annual review of medicine. 2013; 64:357-66.
15. Faul C, Asanuma K, Yanagida-Asanuma E, Kim K, Mundel P. Actin up: regulation of podocyte structure and function by components of the actin cytoskeleton. Trends in cell biology. 2007; 17(9):428-37.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method, comprising two, three, or all four of the following in any order:
determining a level of soluble urokinase-type plasminogen activator receptor (suPAR) protein in a plasma sample from a subject;
determining a level of interleukin 6 (IL-6) protein in a urine sample from the same subject;
determining a level of low molecular weight suPAR in a plasma sample from the subject; and
determining a level of β3 integrin activation activity in a plasma sample from the subject.

2. A method of detecting the presence of suPAR-β3 integrin driven kidney disease in a subject, the method comprising
(a) determining a subject level of two, three, or all four of the following markers:
soluble urokinase-type plasminogen activator receptor (suPAR) protein in a plasma sample from the subject;
interleukin 6 (IL-6) protein in a urine sample from the subject;
low molecular weight suPAR in a plasma sample from the subject; and
β3 integrin activation activity in a plasma sample from the subject;
(b) comparing the subject level of the marker to a reference level; and
(c) detecting the presence of suPAR-β3 integrin driven kidney disease in a subject who has at least two markers above the level.

3. A method of treating a subject who has chronic kidney disease, the method comprising
(a) determining a subject level of two, three, or all four of the following markers:
soluble urokinase-type plasminogen activator receptor (suPAR) protein in a plasma sample from the subject;
interleukin 6 (IL-6) protein in a urine sample from the subject;
low molecular weight suPAR in a plasma sample from the subject; and
β3 integrin activation activity in a plasma sample from the subject;
(b) comparing the subject level of the marker to a reference level; and
(c) selecting and optionally administering a treatment for suPAR-β3 integrin driven kidney disease to a subject who has at least two markers above the level.

4. The method of claim 1, wherein detecting β3 integrin activation activity in a plasma sample comprises contacting the plasma sample with cultured human podocytes in vitro and determining a level of β3 integrin activation in the sample.

5. The method of claim 4, wherein determining a level of β3 integrin activation in the sample comprises contacting the sample with an antibody that binds to beta 3 integrin and an antibody that binds to paxillin and dividing the number of cells expressing beta 3 integrin by the number of cells expressing paxillin.

6. The method of claim 1, wherein the subject has chronic kidney disease.

7. The method of claim 3, wherein the reference value is serum suPAR of ≥3 ng/ml (by ELISA assay); β3 integrin activation >1.2 (AP5/paxillin ratio normalized to healthy serum); presence of low molecular weight suPAR in serum; detectable presence of IL6 in the urine.

8. The method of claim 1, further comprising determining a score calculated using the following algorithm:
score=α×(serum suPAR)+β×(β3 integrin activation)+γ×(low molecular weight suPAR)+δ×(urine IL-6),
wherein each of α, β, γ, and δ are empirically determined weights.

9. The method of claim 8, wherein the algorithm is:
score=0.253×(serum suPAR)+0.282×(β3 integrin activation)+0.212×(low molecular weight suPAR)+0.253×(urine IL-6).

10. The method of claim 3, wherein the treatment for suPAR-β3 integrin driven kidney disease is an α5β3 inhibitor and/or ex vivo removal of suPAR from the subject's circulation.

11. The method of claim 9, wherein the α5β3 inhibitor is a monoclonal antibody that binds specifically to α5β3; a peptide comprising a RGD binding sequence; or a small molecule α5β3 inhibitor.

12. The method of claim 10, wherein the small molecule α5β3 inhibitor is a compound of the formula
or a pharmaceutically acceptable salt thereof.

13. The method of claim 9, wherein the monoclonal antibody that binds specifically to α5β3 is VPI-2960B, CNTO95, or anti-CD61.
